SEQUENCE LISTING (92 sequences)

SEQ ID NO: 1 - DNA, 558 bp, synthetic construct
  misc_feature 1..558: note = MS-B1
  source 1..558: mol_type = other DNA
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, synthetic construct
  REGION 1..184: note = MS-B1
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 885 bp, synthetic construct
  misc_feature 1..885: note = alpha-HL-NN
  source 1..885: mol_type = other DNA
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaaaactatatgagtactttaacttatggattcaacggtaatgttactggtgatgatacaggaaaaattggcggccttattggtgcaaatgtttcgattggtcatacactgaactatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaattaa

SEQ ID NO: 4 - protein, 293 aa, synthetic construct
  REGION 1..293: note = alpha-HL-NN
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYMSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - protein, 184 aa, synthetic construct
  REGION 1..184: note = MspB
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGPAGGVAVSNAHGTVTGAAGGVLLRPFARLIASTGDSVTTYGEPWNMN

SEQ ID NO: 6 - protein, 184 aa, synthetic construct
  REGION 1..184: note = MspC
GLDNELSLVDGQDRTLTVQQWDTFLNGVFPLDRNRLTREWFHSGRAKYIVAGPGADEFEGTLELGYQIGFPWSLGVGINFSYTTPNILIDDGDITGPPFGLESVITPNLFPGVSISADLGNGPGIQEVATFSVDVSGPAGGVAVSNAHGTVTGAAGGVLLRPFARLIASTGDSVTTYGEPWNMN

SEQ ID NO: 7 - protein, 183 aa, synthetic construct
  REGION 1..183: note = MspD
VDNQLSVVDGQGRTLTVQQAETFLNGVFPLDRNRLTREWFHSGRATYHVAGPGADEFEGTLELGYQVGFPWSLGVGINFSYTTPNILIDGGDITQPPFGLDTIITPNLFPGVSISADLGNGPGIQEVATFSVDVKGAKGAVAVSNAHGTVTGAAGGVLLRPFARLIASTGDSVTTYGEPWNMN

SEQ ID NO: 8 - DNA, 1830 bp, synthetic construct
  misc_feature 1..1830: note = Phi29 DNA polymerase
  source 1..1830: mol_type = other DNA
atgaaacacatgccgcgtaaaatgtatagctgcgcgtttgaaaccacgaccaaagtggaagattgtcgcgtttgggcctatggctacatgaacatcgaagatcattctgaatacaaaatcggtaacagtctggatgaatttatggcatgggtgctgaaagttcaggcggatctgtacttccacaacctgaaatttgatggcgcattcattatcaactggctggaacgtaatggctttaaatggagcgcggatggtctgccgaacacgtataataccattatctctcgtatgggccagtggtatatgattgatatctgcctgggctacaaaggtaaacgcaaaattcataccgtgatctatgatagcctgaaaaaactgccgtttccggtgaagaaaattgcgaaagatttcaaactgacggttctgaaaggcgatattgattatcacaaagaacgtccggttggttacaaaatcaccccggaagaatacgcatacatcaaaaacgatatccagatcatcgcagaagcgctgctgattcagtttaaacagggcctggatcgcatgaccgcgggcagtgatagcctgaaaggtttcaaagatatcatcacgaccaaaaaattcaaaaaagtgttcccgacgctgagcctgggtctggataaagaagttcgttatgcctaccgcggcggttttacctggctgaacgatcgtttcaaagaaaaagaaattggcgagggtatggtgtttgatgttaatagtctgtatccggcacagatgtacagccgcctgctgccgtatggcgaaccgatcgtgttcgagggtaaatatgtttgggatgaagattacccgctgcatattcagcacatccgttgtgaatttgaactgaaagaaggctatattccgaccattcagatcaaacgtagtcgcttctataagggtaacgaatacctgaaaagctctggcggtgaaatcgcggatctgtggctgagtaacgtggatctggaactgatgaaagaacactacgatctgtacaacgttgaatacatcagcggcctgaaatttaaagccacgaccggtctgttcaaagatttcatcgataaatggacctacatcaaaacgacctctgaaggcgcgattaaacagctggccaaactgatgctgaacagcctgtatggcaaattcgcctctaatccggatgtgaccggtaaagttccgtacctgaaagaaaatggcgcactgggttttcgcctgggcgaagaagaaacgaaagatccggtgtataccccgatgggtgttttcattacggcctgggcacgttacacgaccatcaccgcggcccaggcatgctatgatcgcattatctactgtgataccgattctattcatctgacgggcaccgaaatcccggatgtgattaaagatatcgttgatccgaaaaaactgggttattgggcccacgaaagtacgtttaaacgtgcaaaatacctgcgccagaaaacctacatccaggatatctacatgaaagaagtggatggcaaactggttgaaggttctccggatgattacaccgatatcaaattcagtgtgaaatgcgccggcatgacggataaaatcaaaaaagaagtgaccttcgaaaacttcaaagttggtttcagccgcaaaatgaaaccgaaaccggtgcaggttccgggcggtgtggttctggtggatgatacgtttaccattaaatctggcggtagtgcgtggagccatccgcagttcgaaaaaggcggtggctctggtggcggttctggcggtagtgcctggagccacccgcagtttgaaaaataataa

SEQ ID NO: 9 - protein, 608 aa, synthetic construct
  REGION 1..608: note = Phi29 DNA polymerase
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 10 - DNA, 1390 bp, synthetic construct
  misc_feature 1..1390: note = WT EcoExo I
  source 1..1390: mol_type = other DNA
atgatgaacgatggcaaacagcagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgc

SEQ ID NO: 11 - protein, 485 aa, synthetic construct
  REGION 1..485: note = WT EcoExo I
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 12 - DNA, 804 bp, synthetic construct
  misc_feature 1..804: note = exonuclease III enzyme from E. coli
  source 1..804: mol_type = other DNA
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 13 - protein, 268 aa, synthetic construct
  REGION 1..268: note = exonuclease III enzyme from E. coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 14 - DNA, 1275 bp, synthetic construct
  misc_feature 1..1275: note = TthRecJ-cd
  source 1..1275: mol_type = other DNA
atgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctg

SEQ ID NO: 15 - protein, 425 aa, synthetic construct
  REGION 1..425: note = TthRecJ-cd
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 16 - DNA, 738 bp, synthetic construct
  misc_feature 1..738: note = bacteriophage lambda exo (redX)
  source 1..738: mol_type = other DNA
tccggaagcggctctggtagtggttctggcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatctggctctggttccggcagcggttccgga

SEQ ID NO: 17 - protein, 226 aa, synthetic construct
  REGION 1..226: note = bacteriophage lambda exo (redX)
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 18 - protein, 760 aa, synthetic construct
  REGION 1..760: note = Hel308 Mbu
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 19 - protein, 707 aa, synthetic construct
  REGION 1..707: note = Hel308 Csy
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 20 - protein, 720 aa, synthetic construct
  REGION 1..720: note = Hel308 Tga
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 21 - protein, 799 aa, synthetic construct
  REGION 1..799: note = Hel308 Mhu
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 22 - protein, 1756 aa, synthetic construct
  REGION 1..1756: note = TraI Eco
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 23 - protein, 726 aa, synthetic construct
  REGION 1..726: note = XPD Mbu
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 24 - protein, 439 aa, synthetic construct
  REGION 1..439: note = Dda 1993
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 25 - protein, 970 aa, synthetic construct
  REGION 1..970: note = TrwC Cba
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 26 - DNA, 786 bp, synthetic construct
  misc_feature 1..786: note = WT CsgG from E. coli Str. K-12 substr. MC4100
  source 1..786: mol_type = other DNA
tgtctgaccgcaccgccgaaagaagcggcacgtccgaccctgatgccgcgtgcacagtcttataaagatctgacccatctgccggctccgacgggcaaaatttttgttagcgtctataacatccaggacgaaaccggtcaatttaaaccgtacccggcgagtaatttctccacggccgttccgcagagtgcaaccgctatgctggtcacggcactgaaagattcccgttggttcattccgctggaacgccagggcctgcaaaacctgctgaatgaacgtaaaattatccgcgcagctcaggaaaacggtaccgtggccattaacaatcgtattccgctgcaaagcctgaccgccgcaaacatcatggttgaaggctctatcatcggttacgaatcaaacgtcaaatcgggcggtgtgggcgcacgttattttggcattggtgctgatacccagtaccaactggaccagatcgcagttaacctgcgcgtggttaatgtcagcaccggcgaaattctgagctctgtgaataccagcaaaacgatcctgtcttacgaagtgcaggctggtgtttttcgtttcattgattatcaacgcctgctggaaggcgaagtcggttacacctcaaacgaaccggtgatgctgtgtctgatgtcggcgattgaaacgggtgttattttcctgatcaatgatggcatcgaccgtggtctgtgggatctgcagaacaaagccgaacgtcaaaatgacattctggtgaaataccgccacatgagtgttccgccggaatcc

SEQ ID NO: 27 - protein, 262 aa, synthetic construct
  REGION 1..262: note = WT CsgG from E. coli Str. K-12 substr. MC4100
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 28 - DNA, 46 bp, synthetic construct
  misc_feature 1..46: note = DNA sequence 1 corrected
  misc_feature 46: note = thymine attached to a 4 iSpC3; C3 phosphoramidite spacer, which is joined to another nucleic acid
  source 1..46: mol_type = other DNA
tttttttttttttttttttttttttttttttttttttttttttttt

SEQ ID NO: 29 - DNA, 3589 bp, synthetic construct
  misc_feature 1..3589: note = DNA sequence 2 corrected
  source 1..3589: mol_type = other DNA
gtgcttacggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggcagcgactaacaaacacaatctgatggc

SEQ ID NO: 30 - DNA, 46 bp, synthetic construct; identical to SEQ ID NO: 28
  misc_feature 1..46: note = DNA sequence 1 as in priority document
  misc_feature 46: note = thymine attached to a 4 iSpC3; C3 phosphoramidite spacer, which is joined to another nucleic acid
  source 1..46: mol_type = other DNA

SEQ ID NO: 31 - DNA, 3525 bp, synthetic construct
  misc_feature 1..3525: note = DNA sequence 2 as in priority document
  source 1..3525: mol_type = other DNA
ggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggc

SEQ ID NO: 32 - DNA, 28 bp, synthetic construct
  misc_feature 1..28: note = DNA sequence 3
  misc_feature 28: note = cytosine attached to a 6 iSp18; 18-atom hexa-ethyleneglycol spacer, which is joined to another nucelic acid
  source 1..28: mol_type = other DNA
gcaatatcagcaccaacagaaacaacct

SEQ ID NO: 33 - DNA, 49 bp, synthetic construct
  misc_feature 1..49: note = DNA sequence 4
  misc_feature 49: note = thymine attached to a 4 iSpC3; C3 phosphoramidite spacer, which is joined to another nucleic acid
  source 1..49: mol_type = other DNA
cgttctgtttatgtttcttgtttgttagccttttttttttttttttttt

SEQ ID NO: 34 - skipped

SEQ ID NO: 35 - DNA, 115 bp, synthetic construct
  misc_feature 1..115: note = AE186
  source 1..115: mol_type = other DNA
caaataagaacattatgatcagtagggctaacaaacaagaaacataaacagaacgtgcttacggttcactactcacgacgatgttttttttggtaccttttttttcaccggaaag

SEQ ID NO: 36 - DNA, 26 bp, synthetic construct
  misc_feature 1..26: note = TE60 corrected
  misc_feature 26: note = thymine attached to a 6 iSp18; 18-atom hexa-ethyleneglycol spacer, which is joined to another nucleic acid
  source 1..26: mol_type = other DNA
ctactgatcataatgttcttatttgt

SEQ ID NO: 37 - DNA, 15 bp, synthetic construct
  misc_feature 1..15: note = TE60 as in priority document
  misc_feature 15: note = thymine attached to a 6 iSp18; 18-atom hexa-ethyleneglycol spacer, which is joined to another nucelic acid
  source 1..15: mol_type = other DNA
gcaatatcagcacct

SEQ ID NO: 38-42 - skipped (5 entries)

SEQ ID NO: 43 - DNA, 40 bp, synthetic construct
  misc_feature 1..40: note = AE263
  source 1..40: mol_type = other DNA
gggtccgggaatttctcagcctgggtcattgaagcctgcc

SEQ ID NO: 44 - DNA, 40 bp, synthetic construct
  misc_feature 1..40: note = AE264
  source 1..40: mol_type = other DNA
ccaaatgattgaacaaattaacatcgctcttgagcaaaaa

SEQ ID NO: 45 - DNA, 40 bp, synthetic construct
  misc_feature 1..40: note = AE265
  source 1..40: mol_type = other DNA
gaacccgcagaacaacaacccgcaacatccgctttcctaa

SEQ ID NO: 46 - DNA, 40 bp, synthetic construct
  misc_feature 1..40: note = AE266
  source 1..40: mol_type = other DNA
cgacactgaatacggggcaacctcatgtcaacgaagaaca

SEQ ID NO: 47 - DNA, 40 bp, synthetic construct
  misc_feature 1..40: note = AE267
  source 1..40: mol_type = other DNA
tcaacttaacgtaaaaacaacttcagacaatacaaatcag

SEQ ID NO: 48 - DNA, 40 bp, synthetic construct
  misc_feature 1..40: note = AE268
  source 1..40: mol_type = other DNA
taatcaattatgacgcaggtatcgtattaattgatctgca

SEQ ID NO: 49 - DNA, 40 bp, synthetic construct
  misc_feature 1..40: note = AE269
  source 1..40: mol_type = other DNA
catatcacaacgtgcgtggaggccatcaaaccacgtcaaa

SEQ ID NO: 50 - DNA, 40 bp, synthetic construct
  misc_feature 1..40: note = AE270
  source 1..40: mol_type = other DNA
caccggaaaggacccgtaaagtgataatgattatcatcta

SEQ ID NO: 51 - DNA, 40 bp, synthetic construct
  misc_feature 1..40: note = AE271
  source 1..40: mol_type = other DNA
tcactactcacgacgatgttttttttggtacctttttttt

SEQ ID NO: 52 - DNA, 40 bp, synthetic construct
  misc_feature 1..40: note = AE272
  source 1..40: mol_type = other DNA
ggctaacaaacaagaaacataaacagaacgtgcttacggt

SEQ ID NO: 53 - DNA, 20 bp, synthetic construct
  misc_feature 1..20: note = AE273
  source 1..20: mol_type = other DNA
ctgggtcattgaagcctgcc

SEQ ID NO: 54 - DNA, 20 bp, synthetic construct
  misc_feature 1..20: note = AE274
  source 1..20: mol_type = other DNA
gggtccgggaatttctcagc

SEQ ID NO: 55 - DNA, 20 bp, synthetic construct
  misc_feature 1..20: note = AE275
  source 1..20: mol_type = other DNA
acatcgctcttgagcaaaaa

SEQ ID NO: 56 - DNA, 20 bp, synthetic construct
  misc_feature 1..20: note = AE276
  source 1..20: mol_type = other DNA
ccaaatgattgaacaaatta

SEQ ID NO: 57 - DNA, 20 bp, synthetic construct
  misc_feature 1..20: note = AE277
  source 1..20: mol_type = other DNA
cgcaacatccgctttcctaa

SEQ ID NO: 58 - DNA, 20 bp, synthetic construct
  misc_feature 1..20: note = AE278
  source 1..20: mol_type = other DNA
gaacccgcagaacaacaacc

SEQ ID NO: 59 - DNA, 20 bp, synthetic construct
  misc_feature 1..20: note = AE279
  source 1..20: mol_type = other DNA
cctcatgtcaacgaagaaca

SEQ ID NO: 60 - DNA, 20 bp, synthetic construct
  misc_feature 1..20: note = AE280
  source 1..20: mol_type = other DNA
cgacactgaatacggggcaa

SEQ ID NO: 61 - DNA, 20 bp, synthetic construct
  misc_feature 1..20: note = AE281
  source 1..20: mol_type = other DNA
cttcagacaatacaaatcag

SEQ ID NO: 62 - DNA, 20 bp, synthetic construct
  misc_feature 1..20: note = AE282
  source 1..20: mol_type = other DNA
tcaacttaacgtaaaaacaa

SEQ ID NO: 63 - DNA, 20 bp, synthetic construct
  misc_feature 1..20: note = AE283
  source 1..20: mol_type = other DNA
atcgtattaattgatctgca

SEQ ID NO: 64 - DNA, 20 bp, synthetic construct
  misc_feature 1..20: note = AE284
  source 1..20: mol_type = other DNA
taatcaattatgacgcaggt

SEQ ID NO: 65 - DNA, 20 bp, synthetic construct
  misc_feature 1..20: note = AE285
  source 1..20: mol_type = other DNA
ggccatcaaaccacgtcaaa

SEQ ID NO: 66 - DNA, 20 bp, synthetic construct
  misc_feature 1..20: note = AE286
  source 1..20: mol_type = other DNA
catatcacaacgtgcgtgga

SEQ ID NO: 67 - DNA, 20 bp, synthetic construct
  misc_feature 1..20: note = AE287
  source 1..20: mol_type = other DNA
gtgataatgattatcatcta

SEQ ID NO: 68 - DNA, 20 bp, synthetic construct
  misc_feature 1..20: note = AE288
  source 1..20: mol_type = other DNA
caccggaaaggacccgtaaa

SEQ ID NO: 69 - DNA, 20 bp, synthetic construct
  misc_feature 1..20: note = AE289
  source 1..20: mol_type = other DNA
ttttttggtacctttttttt

SEQ ID NO: 70 - DNA, 20 bp, synthetic construct
  misc_feature 1..20: note = AE290
  source 1..20: mol_type = other DNA
tcactactcacgacgatgtt

SEQ ID NO: 71 - DNA, 20 bp, synthetic construct
  misc_feature 1..20: note = AE291
  source 1..20: mol_type = other DNA
aaacagaacgtgcttacggt

SEQ ID NO: 72 - DNA, 20 bp, synthetic construct
  misc_feature 1..20: note = AE292
  source 1..20: mol_type = other DNA
ggctaacaaacaagaaacat

SEQ ID NO: 73 - DNA, 80 bp, synthetic construct
  misc_feature 1..80: note = AE258
  source 1..80: mol_type = other DNA
ccaaatgattgaacaaattaacatcgctcttgagcaaaaagggtccgggaatttctcagcctgggtcattgaagcctgcc

SEQ ID NO: 74 - DNA, 80 bp, synthetic construct
  misc_feature 1..80: note = AE259
  source 1..80: mol_type = other DNA
cgacactgaatacggggcaacctcatgtcaacgaagaacagaacccgcagaacaacaacccgcaacatccgctttcctaa

SEQ ID NO: 75 - DNA, 90 bp, synthetic construct
  misc_feature 1..90: note = AE182
  source 1..90: mol_type = other DNA
atctgcactgctcattaatatacttctgggttccttccagttgtttttgcatagtgatcagcctctctctgagggtgaaataatcccgtt

SEQ ID NO: 76 - DNA, 48 bp, synthetic construct
  misc_feature 1..48: note = TH14
  misc_feature 1: note = is 5' phosphorylated
  misc_feature 48: note = cytosine attached to a 4 iSp18; 18-atom hexa-ethyleneglycol spacer, which is joined to another nucleic acid
  source 1..48: mol_type = other DNA
cgtggttggtgtggttggcggacactgattgacacggtttagtagagc

SEQ ID NO: 77 - DNA, 48 bp, synthetic construct
  misc_feature 1..48: note = TH15
  misc_feature 1: note = is 5' phosphorylated
  misc_feature 48: note = cytosine attached to a 4 iSp18; 18-atom hexa-ethyleneglycol spacer, which is joined to another nucelic acid
  source 1..48: mol_type = other DNA
cgtccaaccacaccaacccggacactgattgacacggtttagtagagc

SEQ ID NO: 78 - DNA, 61 bp, synthetic construct
  misc_feature 1..61: note = TH16
  misc_feature 1: note = is 5' phosphorylated
  misc_feature 61: note = cytosine attahced to a 4 iSp18; 18-atom hexa-ethyleneglycol spacer, which is joined to another nucleic acid
  source 1..61: mol_type = other DNA
cgtagtccgtggtagggcaggttggggtgaccggacactgattgacacggtttagtagagc

SEQ ID NO: 79 - DNA, 62 bp, synthetic construct
  misc_feature 1..62: note = TH17
  misc_feature 1: note = is 5' phosphorylated
  misc_feature 62: note = cytosine attached to a 4 iSp18; 18-atom hexa-ethyleneglycol spacer,which is joined to another nucleic acid
  source 1..62: mol_type = other DNA
cgtagtcaccccaacctgccctaccacggactcggacactgattgacacggtttagtagagc

SEQ ID NO: 80 - DNA, 48 bp, synthetic construct
  misc_feature 1..48: note = Control hairpin sequence
  misc_feature 1: note = is 5' phosphorylated
  misc_feature 48: note = cytosine attached to a 4 iSpC3; C3 phosphoramidite spacer, which is joined to another nucleic acid
  source 1..48: mol_type = other DNA
cgtcctgtcgctgtgtctcggacactgattgacacggtttagtagagc

SEQ ID NO: 81 - DNA, 35 bp, synthetic construct
  misc_feature 1..35: note = Y adapter sequence 1
  misc_feature 1: note = guanine attached to a 30 iSpC3; C3 phosphoramidite spacer
  misc_feature 35: note = thymine attached to a 4 iSp18; 18-atom hexa-ethyleneglycol spacer, which is joined to another nucleic acid
  source 1..35: mol_type = other DNA
ggcgtctgcttgggtgtttaacctttttttttttt

SEQ ID NO: 82 - DNA, 45 bp, synthetic construct
  misc_feature 1..45: note = Y adapter sequence 2
  misc_feature 1: note = is 5' phosphorylated
  modified_base 23: mod_base = OTHER; note = is iBNA-MeC, where BNA is Bridged Nucleic Acid, where MeC is methyl cytosine
  modified_base 24: mod_base = OTHER; note = is iBNA-A, where BNA is Bridged Nucleic Acid
  misc_feature 25: note = is iBNA-A, where BNA is Bridged Nucleic Acid
  modified_base 26: mod_base = OTHER; note = is iBNA-MeC, where BNA is Bridged Nucleic Acid, where MeC is metylcytosine
  modified_base 27: mod_base = OTHER; note = is iBNA-MeC, where BNA is Bridged Nucleic Acid, where MeC is metylcytosine
  source 1..45: mol_type = other DNA
gcaatatcagcaccaacagaaacaacctttgaggcgagcggtcaa

SEQ ID NO: 83 - DNA, 25 bp, synthetic construct
  misc_feature 1..25: note = Y adapter sequence 3
  modified_base 1: mod_base = OTHER; note = is iBNA-G, where BNA is Bridged Nucleic Acid
  modified_base 2: mod_base = OTHER; note = is iBNA-G, where BNA is Bridged Nucleic Acid
  modified_base 3: mod_base = OTHER; note = is iBNA-T, where BNA is Bridged Nucleic Acid
  modified_base 4: mod_base = OTHER; note = is iBNA-T, where BNA is Bridged Nucleic Acid
  modified_base 5: mod_base = OTHER; note = is iBNA-A, where BNA is Bridged Nucleic Acid
  source 1..25: mol_type = other DNA
ggttaaacacccaagcagacgcctt

SEQ ID NO: 84 - DNA, 3620 bp, synthetic construct
  source 1..3620: mol_type = other DNA
  modified_base 28..31: mod_base = OTHER; note = Modified by 4 i5NitInd, also known as 5-Nitroindole or universal base
  misc_feature 1..3620: note = DNA sequence 1 corrected
  misc_feature 1: note = guanine attached to a 4 iSpC3; C3 phosphoramidite spacer, which is joined to another nucleic acid
ggttgtttctgttggtgctgatattgcnnnngccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcac

SEQ ID NO: 85 - DNA, 3530 bp, synthetic construct
  source 1..3530: mol_type = other DNA
  modified_base 28..31: mod_base = OTHER; note = Modified by 4 i5NitInd, also known as 5-Nitroindole or universal base
  misc_feature 1..3530: note = DNA sequence 1 as in priority document
  misc_feature 1: note = guanine attached to a 4 iSpC3; C3 phosphoramidite spacer, which is joined to another nucleic acid
ggttgtttctgttggtgctgatattgcnnnngccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacggg

SEQ ID NO: 86 - DNA, 35 bp, synthetic construct
  source 1..35: mol_type = other DNA
  misc_feature 1: note = thymine attached to a 4 iSpC3; C3 phosphoramidite spacer, which is joined to another nucleic acid
  misc_feature 1..35: note = DNA sequence 4
tttttggctaacaaacaagaaacataaacagaacg

SEQ ID NO: 87 - DNA, 46 bp, synthetic construct
  source 1..46: mol_type = other DNA
  misc_feature 1: note = thymine attached to a 4 iSp18; 18-atom hexa-ethyleneglycol spacer, which is joined to another nucleic acid
  misc_feature 1..46: note = TH14
tttttttttttttttttttttttcgccaaccacaccaaccacgtcc

SEQ ID NO: 88 - DNA, 47 bp, synthetic construct
  source 1..47: mol_type = other DNA
  misc_feature 1: note = thymine attached to a 4 iSp18; 18-atom hexa-ethyleneglycol spacer, which is joined to another nucleic acid
  misc_feature 1..47: note = TH15
tttttttttttttttttttttttcgggttggtgtggttggacgtcct

SEQ ID NO: 89 - DNA, 61 bp, synthetic construct
  source 1..61: mol_type = other DNA
  misc_feature 1: note = thymine attahced to a 4 iSp18; 18-atom hexa-ethyleneglycol spacer, which is joined to another nucleic acid
  misc_feature 1..61: note = TH16
tttttttttttttttttttttttcgagtcaccccaacctgccctaccacggactacgtcct

SEQ ID NO: 90 - DNA, 61 bp, synthetic construct
  source 1..61: mol_type = other DNA
  misc_feature 1: note = thymine attached to a 4 iSp18; 18-atom hexa-ethyleneglycol spacer,which is joined to another nucleic acid
  misc_feature 1..61: note = TH17
tttttttttttttttttttttttcgagtccgtggtagggcaggttggggtgactacgtcct

SEQ ID NO: 91 - DNA, 52 bp, synthetic construct
  source 1..52: mol_type = other DNA
  misc_feature 1: note = thymine attached to a 4 iSpC3; C3 phosphoramidite spacer, which is joined to another nucleic acid
  misc_feature 1..52: note = Control hairpin sequence
ttttttttttttttttttttttttttttcgagacacagcgacaggacgtcct

SEQ ID NO: 92 - DNA, 28 bp, synthetic construct
  source 1..28: mol_type = other DNA
  misc_feature 1: note = guanine attached to a 4 iSp18; 18-atom hexa-ethyleneglycol spacer, which is joined to another nucleic acid
  misc_feature 1..28: note = Y adapter sequence 1
ggttgtttctgttggtgctgatattgct

(19) United States
(12) Patent Application Publication
(10) Pub. No.: US 2025/0207188 A1
(43) Pub. Date: Jun. 26, 2025

(54) METHOD

(71) Applicant: Oxford Nanopore Technologies PLC, Oxford (GB)

(72) Inventors: Richard Alexander Gutierrez, Oxford (GB); Andrew John Heron, Oxford (GB); James White, Oxford (GB)

(73) Assignee: Oxford Nanopore Technologies PLC, Oxford (GB)

(21) Appl. No.: 18/989,888

(22) Filed: Dec. 20, 2024

Related U.S. Application Data
(63) Continuation of application No. 16/304,077, filed on Nov. 21, 2018, now Pat. No. 12,215,384.

(30) Foreign Application Priority Data
May 25, 2016 (GB) 1609241.3
May 27, 2016 (GB) 1609436.9

(51) Int. Cl. C12Q 1/6869

(52) U.S. Cl. CPC C12Q 1/6869; C12Q 2565/631

(57) ABSTRACT

The invention relates to a new method of characterising a target polynucleotide using a pore. The method involves controlling the formation of secondary structure by the target polynucleotide after the polynucleotide has moved through the pore.

### RELATED APPLICATIONS

This application is a continuation of U.S. application Ser. No. 16/304,077, filed Nov. 21, 2018, which is a national stage filing under 35 U.S.C. § 371 of international application number PCT/GB2017/051491, filed May 25, 2017, and claims the benefit under 35 U.S.C. § 119 (e) of GB application numbers 1609241.3, filed May 25, 2016, and 1609436.9, filed May 27, 2016, each of which is herein incorporated by reference in its entirety.

### REFERENCE TO AN ELECTRONIC SEQUENCE LISTING

The contents of the electronic sequence listing (0036670066US01-SUBSEQ-FL.xml; Size: 162,680 bytes; and Date of Creation: Feb. 28, 2025) are herein incorporated by reference in its entirety.

### FIELD OF THE INVENTION

The invention relates methods of characterising a target polynucleotide using a pore.

### BACKGROUND TO THE INVENTION

There is currently a need for rapid and cheap polynucleotide (e.g. DNA or RNA) sequencing and identification technologies across a wide range of applications. Existing technologies are slow and expensive mainly because they rely on amplification techniques to produce large volumes of polynucleotide and require a high quantity of specialist fluorescent chemicals for signal detection.

Transmembrane pores (nanopores) have great potential as direct, electrical biosensors for polymers and a variety of small molecules. In particular, recent focus has been given to nanopores as a potential DNA sequencing technology.

When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing method, a single polynucleotide strand is passed through the pore and the identities of the nucleotides are derived. Strand sequencing can involve the use of a molecular brake to control the movement of the polynucleotide through the pore.

### SUMMARY OF THE INVENTION

The inventors have surprisingly demonstrated that it is possible to improve the characterisation of a target polynucleotide by controlling the formation of secondary structure by the polynucleotide after it has moved through a transmembrane pore.

Accordingly, a method of characterising a target polynucleotide is provided, which comprises:
(a) contacting the target polynucleotide with one side of a transmembrane pore in a membrane and a molecular brake which controls the movement of the target polynucleotide through the pore; and
(b) taking one or more measurements which are indicative of one or more characteristics of the target polynucleotide as the polynucleotide moves with respect to the pore; wherein the conditions on the other side of the pore are selected to control the formation of secondary structure by the target polynucleotide on the other side of the pore.

Also provided is a method of characterising a double stranded target polynucleotide, comprising:
(a) providing a construct comprising the target polynucleotide in which the two strands of the target polynucleotide are linked at one end of the target polynucleotide by a hairpin loop;
(b) contacting the construct with one side of a transmembrane pore in a membrane and a molecular brake which separates the two strands of the construct and controls the movement of the construct through the pore one strand at a time; and
(c) taking one or more measurements which are indicative of one or more characteristics of the target polynucleotide as the construct moves with respect to the pore; wherein the hairpin loop is designed to control the ability of the two strands of the target polynucleotide to rehybridise on the other side of the pore.

Controlling the ability of the two strands of the target polynucleotide to rehybridise may may control the extent and/or consistency of formation of rehybridised polynucleotide (e.g. the proportion of polynucleotides that pass through the pore that rehybririse and the reproducibility of hybridisation, for example, in terms of how quickly it happens, how strong the binding is).

Further provided are:
a kit for characterising a double stranded target polynucleotide comprising (a) a hairpin loop capable of linking the two strands of the target polynucleotide at one end and (b) one or more species which control or decrease the formation of secondary structure by the target polynucleotide; and
an apparatus for sequencing a target polynucleotide, comprising: (a) a plurality of membranes; (b) a plurality of transmembrane pores in the membranes; and (c) conditions on the other side of the pores from which the pores are contacted with the polynucleotide which are capable of controlling the formation of secondary structure by the target polynucleotide.

### DESCRIPTION OF THE FIGURES

FIG. 1 shows a schematic of construct 1 where A is a helicase (T4 Dda 1993-(E94C/A360C) (SEQ ID NO: 24 with mutations E94C/A360C and then (ΔM1) G1G2 (where (ΔM1) G1G2=deletion of M1 and then addition G1 and G2)) bound on the indicated region of DNA sequence 1 (B) which is hybridised to DNA sequence 2 (D) and DNA sequence 3 (E). DNA sequence 1 is also attached to DNA sequence 2 with a ligated hairpin, DNA sequence 4 (C).

FIG. 2 shows an example current trace (y-axis label=Current (pA), x-axis label=Time(s)) of when a helicase (T4 Dda 1993-(E94C/A360C)) controlled the translocation of the DNA construct 1 through an MspA nanopore. The lower picture is a zoomed in version of the upper one. The region labelled A corresponds to the template (DNA sequence 1). The region labelled B corresponds to the complement (DNA sequence 2). The region labelled C corresponds to the hairpin (DNA sequence 4). In this figure, the template and complement have similar current range, are of similar duration and have similar speed. This is an example of a helicase controlled DNA movement exhibiting No Uplift.

FIG. 3 shows an example current trace (y-axis label=Current (pA), x-axis label=Time(s)) of when a helicase (T4 Dda 1993-(E94C/A360C)) controlled the translocation of the DNA construct 1 through an MspA nanopore. The lower picture is a zoomed in version of the upper one. The region labelled A corresponds to the template (DNA sequence 1). The region labelled B corresponds to the complement (DNA sequence 2). The region labelled C corresponds to the hairpin (DNA sequence 4). In this figure, the complement has a higher current range than the template, the duration is shorter and the speed is faster. This is an example of helicase controlled DNA movement exhibiting Uplift.

FIG. 4 shows an example current trace (y-axis label=Current (pA), x-axis label=Time(s)) of when a helicase (T4 Dda 1993-(E94C/A360C)) controlled the translocation of the DNA construct 1 through an MspA nanopore. The lower picture is a zoomed in version of the upper one. The region labelled A corresponds to the template (DNA sequence 1). The regions labelled B1 and B2 correspond to the complement (DNA sequence 2). The region labelled C corresponds to the hairpin (DNA sequence 4). In this figure, the complement initially has a similar current range and speed to the template (B1) and then changes to a behaviour where the current range is higher than the template, the speed is faster and the duration is shorter. This is an example of helicase controlled DNA movement exhibiting Delayed Uplift.

FIG. 5 shows an example current trace (y-axis label=Current (pA), x-axis label=Time(s)) of when a helicase (T4 Dda 1993-(E94C/A360C)) controlled the cis to trans translocation of the DNA construct 1 through an MspA nanopore with a single stranded DNA (AE182) present on the trans side of the membrane. The lower picture is a zoomed in version of the upper one. The region labelled A corresponds to the template (DNA sequence 1). The regions labelled B1 and B2 correspond to the complement (DNA sequence 2). The region labelled C corresponds to the hairpin (DNA sequence 4). In this figure, the complement initially has a higher current range and speed to the template (B1) and then changes to a behaviour where the current range and speed are similar to the template (B2), then goes back to the initial behaviour (B1). The AE182 strand was designed to hybridise to a region in the middle of the template region of construct 1. This data shows that the AE182 controls the formation of secondary structure in the region of construct 1 to which it hybridises.

FIG. 6 shows data acquired by observing helicase (T4 Dda 1993-(E94C/A360C)) controlled translocation of the DNA construct 1 through an MspA nanopore, and categorising the helicase controlled DNA movements into one of the three categories: uplift, no uplift or delayed uplift. The trans DNA (Col 1) and conc. columns (Col 2) show what was present in the trans chamber (the side of the nanopore opposite to which the target polynucleotide was added) in addition to buffer 1.

FIG. 7 shows the data in FIG. 6 as a bar chart. The numbers on the x axis correspond to the row numbers of the data table (FIG. 6). The numbers on the y axis correspond to the % of observed helicase controlled movements. The height of the black bar represents the % of translocations that have been categorised as “no uplift” as opposed to “uplift” or “delayed uplift”. The control conditions (no polynucleotide sequences added to the trans side to control secondary structure) is shown in row number 13 and is marked with an arrow.

FIG. 8 shows an example current trace (y-axis label=Current (pA), x-axis label=Time(s)) of when a helicase (T4 Dda 1993-(E94C/A360C)) controlled the translocation of the DNA construct 1 through an MspA nanopore. The lower pictures are zoomed in versions of the top one. The region labelled A corresponds to the template (DNA sequence 1). The regions labelled B1 and B2 correspond to the complement (DNA sequence 2). The region labelled C corresponds to the hairpin (DNA sequence 4). In this figure, the complement initially has a higher current range and speed to the template (B1) and then changes to a behaviour where the current range and speed are similar to the template. This example trace shows that the salt active nuclease (SAN) cut construct 1 part way along the template region. Thus uplift was observed in the region labelled B1 but not in the region labelled B2 because the region of the template which hybridised to the complement had been cut off by the SAN when region B2 translocated through the nanopore.

FIG. 9 shows a double stranded polynucleotide with a template region, a complement region and a prebound enzyme. The helicase controls the movement of the polynucleotide through the nanopore. Once the hairpin (which connects the template and complement regions) translocates through the nanopore then DNA begins to re-hybridise on the trans side of the nanopore. It is the rehybridisation which is thought to result in uplift being observed in the complement region under helicase controlled DNA movement.

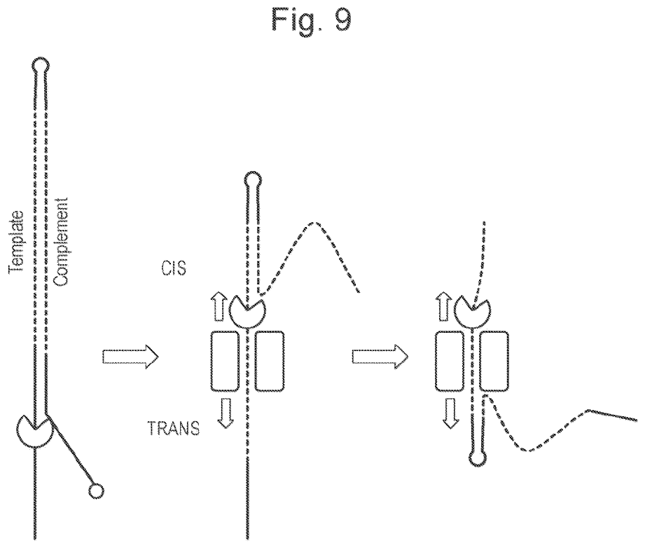

FIG. 10 shows a number of methods which can be used to control the formation of secondary structure on the trans side of the nanopore. A) shows the hybridisation of polynucleotides to the target polynucleotide on the trans side which prevents it reforming into a double-stranded construct with the complement region as it translocates through the nanopore. B) shows the use of protein or chemical traps which bind to the target polynucleotide on the trans side and prevents it reforming into a double-stranded construct with the complement region as it translocates through the nanopore. C) shows the use of a nuclease enzyme to cut the target polynucleotide on the trans side and prevents it reforming into a double-stranded construct with the complement region as it translocates through the nanopore.

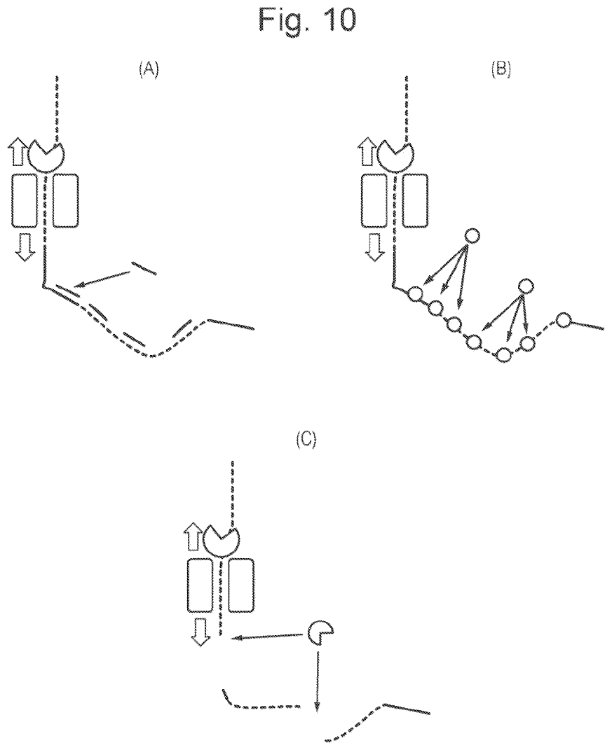

FIG. 11 shows a 4-20% TBE gel showing the quadruplex containing oligos. Col A=oligo TH14 (stock). Col B=TH14 (snapcooled). Col C=TH15 (stock). Col D=TH15 (snapcooled). Col E=TH16 (stock). Col F=TH16 (snapcooled). Col G=TH17 (stock). Col H=TH17 (snapcooled). A single band in the gel shown in FIG. 11 suggests the oligonucleotide is a single species.

FIG. 12 shows a bar chart. The numbers on the x axis correspond the oligo's which have quadruplexes present in their hairpins and were ligated to dsDNA-1=control, 2=TH14, 3=TH15, 4=TH16 and 5=TH17. The numbers on the y axis correspond to the % of observed helicase controlled movements. The height of the lower region of the bar represents the % of translocations that have been categorised as no uplift and the height of the upper region of the bar represents the % of translocations that have been categorized as uplift, i.e. the shorter the upper region of the bar, the lower the uplift. The results for control conditions (no polynucleotide sequences added to the trans side to control secondary structure) are shown in row number 1.

It is to be understood that Figures are for the purpose of illustrating particular embodiments of the invention only, and are not intended to be limiting.

### Description of the Sequence Listing

SEQ ID NO: 1 shows the codon optimised polynucleotide sequence encoding the MS-B1 mutant MspA monomer. This mutant lacks the signal sequence and includes the following mutations: D90N, D91N, D93N, D118R, D134R and E139K.
SEQ ID NO: 2 shows the amino acid sequence of the mature form of the MS-B1 mutant of the MspA monomer. This mutant lacks the signal sequence and includes the following mutations: D90N, D91N, D93N, D118R, D134R and E139K.
SEQ ID NO: 3 shows the polynucleotide sequence encoding one monomer of α-hemolysin-E111N/K147N (α-HL-NN; Stoddart et al., PNAS, 2009; 106 (19): 7702-7707).
SEQ ID NO: 4 shows the amino acid sequence of one monomer of α-HL-NN.
SEQ ID NOs: 5 to 7 show the amino acid sequences of MspB, C and D.
SEQ ID NO: 8 shows the polynucleotide sequence encoding the Phi29 DNA polymerase.
SEQ ID NO: 9 shows the amino acid sequence of the Phi29 DNA polymerase.
SEQ ID NO: 10 shows the codon optimised polynucleotide sequence derived from the sbcB gene from E. coli. It encodes the exonuclease I enzyme (EcoExo I) from E. coli.
SEQ ID NO: 11 shows the amino acid sequence of exonuclease I enzyme (EcoExo I) from E. coli.
SEQ ID NO: 12 shows the codon optimised polynucleotide sequence derived from the xthA gene from E. coli. It encodes the exonuclease III enzyme from E. coli.
SEQ ID NO: 13 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides.
SEQ ID NO: 14 shows the codon optimised polynucleotide sequence derived from the recJ gene from T. thermophilus. It encodes the RecJ enzyme from T. thermophilus (TthRecJ-cd).
SEQ ID NO: 15 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides.
SEQ ID NO: 16 shows the codon optimised polynucleotide sequence derived from the bacteriophage lambda exo (redX) gene. It encodes the bacteriophage lambda exonuclease.
SEQ ID NO: 17 shows the amino acid sequence of the bacteriophage lambda exonuclease. The sequence is one of three identical subunits that assemble into a trimer. The enzyme performs highly processive digestion of nucleotides from one strand of dsDNA, in a 5′-3′ direction (www.neb.com/nebecomm/products/productM0262.asp). Enzyme initiation on a strand preferentially requires a 5′ overhang of approximately 4 nucleotides with a 5′ phosphate.
SEQ ID NO: 18 shows the amino acid sequence of He1308 Mbu.
SEQ ID NO: 19 shows the amino acid sequence of He1308 Csy.
SEQ ID NO: 20 shows the amino acid sequence of He1308 Tga.
SEQ ID NO: 21 shows the amino acid sequence of He1308 Mhu.
SEQ ID NO: 22 shows the amino acid sequence of TraI Eco.
SEQ ID NO: 23 shows the amino acid sequence of XPD Mbu.
SEQ ID NO: 24 shows the amino acid sequence of Dda 1993.
SEQ ID NO: 25 shows the amino acid sequence of Trwc Cba.
SEQ ID NO: 26 shows the codon optimised polynucleotide sequence encoding the wild-type CsgG monomer from Escherichia coli Str. K-12 substr. MC4100. This monomer lacks the signal sequence.
SEQ ID NO: 27 shows the amino acid sequence of the mature form of the wild-type CsgG monomer from Escherichia coli Str. K-12 substr. MC4100. This monomer lacks the signal sequence. The abbreviation used for this CsgG=CsgG-Eco.
SEQ ID NO: 28 is DNA sequence 1 used in Example 1.
SEQ ID NO: 29 is DNA sequence 2 used in Example 1.
SEQ ID NO: 30 is an alternative DNA sequence 1 that could be used in Example 1.
SEQ ID NO: 31 is an alternative DNA sequence 2 that could be used in Example 1.
SEQ ID NO: 32 is DNA sequence 3 used in Example 1.
SEQ ID NO: 33 is DNA sequence 4 used in Example 1.
SEQ ID NO: 34 is the DNA sequence of AE202 used in Example 1.
SEQ ID NO: 35 is the DNA sequence of AE186 used in Example 1.
SEQ ID NO: 36 is the DNA sequence of TE60 used in Example 1.
SEQ ID NO: 37 is an alternative DNA sequence of TE60.
SEQ ID NO: 38 is the DNA sequence of AE203 used in Example 1.
SEQ ID NO: 39 is the DNA sequence of AE210 used in Example 1.
SEQ ID NO: 40 is the DNA sequence of AE191 used in Example 1.
SEQ ID NO: 41 is the DNA sequence of AE192 used in Example 1.
SEQ ID NO: 42 is the DNA sequence of AE193 used in Example 1.
SEQ ID NO: 43 is the DNA sequence of AE263 used in Example 1.
SEQ ID NO: 44 is the DNA sequence of AE264 used in Example 1.
SEQ ID NO: 45 is the DNA sequence of AE265 used in Example 1.
SEQ ID NO: 46 is the DNA sequence of AE266 used in Example 1.
SEQ ID NO: 47 is the DNA sequence of AE267 used in Example 1.
SEQ ID NO: 48 is the DNA sequence of AE268 used in Example 1.
SEQ ID NO: 49 is the DNA sequence of AE269 used in Example 1.
SEQ ID NO: 50 is the DNA sequence of AE270 used in Example 1.
SEQ ID NO: 51 is the DNA sequence of AE271 used in Example 1.
SEQ ID NO: 52 is the DNA sequence of AE272 used in Example 1.
SEQ ID NO: 53 is the DNA sequence of AE273 used in Example 1.
SEQ ID NO: 54 is the DNA sequence of AE274 used in Example 1.
SEQ ID NO: 55 is the DNA sequence of AE275 used in Example 1.
SEQ ID NO: 56 is the DNA sequence of AE276 used in Example 1.
SEQ ID NO: 57 is the DNA sequence of AE277 used in Example 1.
SEQ ID NO: 58 is the DNA sequence of AE278 used in Example 1.
SEQ ID NO: 59 is the DNA sequence of AE279 used in Example 1.
SEQ ID NO: 60 is the DNA sequence of AE280 used in Example 1.
SEQ ID NO: 61 is the DNA sequence of AE281 used in Example 1.
SEQ ID NO: 62 is the DNA sequence of AE282 used in Example 1.
SEQ ID NO: 63 is the DNA sequence of AE283 used in Example 1.
SEQ ID NO: 64 is the DNA sequence of AE284 used in Example 1.
SEQ ID NO: 65 is the DNA sequence of AE285 used in Example 1.
SEQ ID NO: 66 is the DNA sequence of AE286 used in Example 1.
SEQ ID NO: 67 is the DNA sequence of AE287 used in Example 1.
SEQ ID NO: 68 is the DNA sequence of AE288 used in Example 1.
SEQ ID NO: 69 is the DNA sequence of AE289 used in Example 1.
SEQ ID NO: 70 is the DNA sequence of AE290 used in Example 1.
SEQ ID NO: 71 is the DNA sequence of AE291 used in Example 1.
SEQ ID NO: 72 is the DNA sequence of AE292 used in Example 1.
SEQ ID NO: 73 is the DNA sequence of AE258 used in Example 1.
SEQ ID NO: 74 is the DNA sequence of AE259 used in Example 1.
SEQ ID NO: 75 is the DNA sequence of AE182 used in Example 1.
SEQ ID NO: 76 is the DNA sequence of TH14 used in Example 3.
SEQ ID NO: 77 is the DNA sequence of TH15 used in Example 3.
SEQ ID NO: 78 is the DNA sequence of TH16 used in Example 3.
SEQ ID NO: 79 is the DNA sequence of TH17 used in Example 3.
SEQ ID NO: 80 is the DNA sequence of a control hairpin used in Example 3.
SEQ ID NO: 81 is the DNA sequence of Y adaptor sequence 1 used in Example 3.
SEQ ID NO: 82 is the DNA sequence of Y adaptor sequence 2 used in Example 3.
SEQ ID NO: 83 is the DNA sequence of Y adaptor sequence 3 used in Example 3.It is to be understood that sequences are not intended to be limiting.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”,“an”, and “the” include plural referents unless the content clearly dictates otherwise. Thus, for example, reference to “a polynucleotide” includes two or more polynucleotides, reference to “an anchor” refers to two or more anchors, reference to “a helicase” includes two or more helicases, and reference to “a transmembrane pore” includes two or more pores and the like.

In this specification, where different amino acids at a specific position are separated by the symbol “/”, the/symbol “/” means “or”. For instance, P108R/K means P108R or P108K. In this specification where different positions or different substitutions are separated by the symbol “/” “/”, the “/” symbol means “and”. For example, E94/P108 means E94 and P108 or E94D/P108Kmeans E94D and P108K.

All publications, patents and patent applications cited herein, whether supra or infra, are hereby incorporated by reference in their entirety.

### Methods of the Invention

The invention provides an improved method of characterising a target polynucleotide. The target polynucleotide is contacted with one side of a transmembrane pore in a membrane. The target polynucleotide is also contacted with a molecular brake which controls the movement of the target polynucleotide through the pore. Suitable pores, membranes and molecular brakes are discussed in more detail below. One or more measurements which are indicative of one or more characteristics of the target polynucleotide are taken as the polynucleotide moves with respect to the pore. An important part of the invention is that conditions on the other side of the pore (i.e. the other side of the pore from the one side of the pore with which the target polynucleotide is contacted) control the formation of secondary structure by the target polynucleotide on this other side of the pore. In other words, the conditions on the other side of the pore are selected to control the formation of secondary structure by the target polynucleotide after it has moved through the pore. Control of the formation of secondary structure may comprise controlling the extent and/or increasing the consistency to which the secondary structure is formed. Formation of secondary structure can be a random and variable process and control of the extent of formation and/or increasing the consistency of formation of secondary structure lowers the variability or randomness of formation. This can give rise to an improved measurement accuracy as further described below.

It may, for example, be desirable to increase formation of secondary structure such that strong (GC) vs weak (AT) interactions may be distinguished. Since formation of secondary structure can be detected as part of the current trace, sequence specific information could be extracted by doing this. For example, the signal can be used to empirically measure secondary structures that may be hard to measure by conventional means. As a particular example, the signal could be used to empirically measure the presence of such structures G-quadraplex formation.

Another situation in which it is desirable to increase formation of secondary structure is where there is Inconsistent behavior within a strand, giving rise to inconsistent signal. An inconsistent signal is hard to model and therefore can produce lower accuracy base calling. It may therefore be preferable to have a consistent uplifted signal from consistent formation of the secondary structure where the alternative is an inconsistent signal.

The conditions include, for example, voltage, salt type, salt concentration, temperature, presence of DNA and/or pH. Changing any one or more of these conditions will affect polynucleotide hybridization/secondary structures.

Secondary structure formation may be detected and measured by any suitable means. For example, by subjecting a polynucleotide to the condition to be tested and running a melt curve assay using standard procedures known in the art. Secondary structure formation may alternatively be detected and measured by measuring the frequency and magnitude of the uplifted/non-uplifted signal changes as a polynucleotide passing through a nanopore as described in the Examples. The conditions may be selected to control the formation of secondary structure by or in the target polynucleotide in any way and to any extent. The conditions may decrease, reduce or prevent the formation of secondary structure. The conditions may decrease, reduce or prevent the formation of secondary structure by any amount, such by at least 10%, at least 20%, at least 30%, at least 40%, at least 50%, at least 60%, at least 70%, at least 80% or at least 90%. The decrease, reduction or prevention of secondary structure formation is typically compared to a reference condition. The method more preferably abolishes the formation of secondary structure (i.e. decreases, reduces or prevents it by 100%).

Alternatively, the conditions may increase the formation of secondary structure. The conditions may increase the formation of secondary structure by any amount, such by at least 10%, at least 20%, at least 30%, at least 40%, at least 50%, at least 60%, at least 70%, at least 80% or at least 90%. The increase of secondary structure formation is typically compared to a reference condition. The method may increase the formation of secondary structure by at least twofold, at least threefold, at least fourfold, at least fivefold, at least tenfold, at least twentyfold, at least thirtyfold, at least fortyfold, at least fiftyfold, at least one hundredfold, at least five hundredfold or one thousandfold.

The skilled person would readily be able to determine a suitable reference condition depending on the condition being altered to control secondary structure formation. For example, where the condition is the presence of DNA, the reference condition is the absence of the DNA. A typical reference condition is 150 mM potassium ferricyanide, 150 mM potassium ferrocyanide, 25 mM potassium phosphate, pH8 at 34° C.

For any one strand there is a probability that sequence dependent secondary structure will form (0-100% probability, more precisely close to zero to 100% probability, i.e. <<1% to 100%probability). The probability depends on the physical characteristics of a given sequence, including length, sequence, and possibly other factors such as methylation and/or damage. Moreover, for any given sequence a variety of structural elements may form, each with a probability dependent on conditions (total probability of all forms adding up to 100%). For example a given sequence may form: 1) approximately linearized unstructured ssDNA/ssRNA at 20% probability; 2) secondary structure A at 40% probability; 3) secondary structure B at 30%probability; and 4) secondary structure C at 10% probability. Secondary structures A, B and Care different secondary structure conformations. Various secondary structure conformations are known in the art and include, for example, stem loops, hairpins, triplex DNA, pseudoknots, quadruplex structures etc. The formation of secondary structure may be measured in any known way.

“Uplift” is a term used to describe an increase in the current range of a polynucleotide hairpin strand passing through a nanopore from the cis side of a membrane to the trans side of the membrane. It occurs when the second (complement) strand of a double stranded polynucleotide is passing through the nanopore and the first (template) strand of a double stranded polynucleotide is in the trans. Uplift may be observed as an increase in complement signal relative to template signal.

“No uplift” is identified when the current range when the first (template) strand of a double stranded polynucleotide is translocating through a pore is similar, or substantially similar (e.g., the same) as the current range when the second (complement) strand of a double stranded polynucleotide is translocating through the pore. “Non uplift” may be observed when the complement signal is approximately the same as the template signal.

Current range is the difference in current between the lowest levels and highest levels in the signal. This can be defined many ways, e.g. current range=Max current-min current, or range=95^{th} percentile-5^{th} percentile.

Increased current range is said to occur when it can be observed above any normal signal noise/variation, e.g. in the order of >5% or >10%.

Delayed uplift shows that secondary structure is a time-bound variable and inconsistent process, that can occur either immediately as the complement starts exiting the pore, or some time later. In other words “delayed uplift” is when “no uplift” behavior becomes an “uplift” behavior. Mechanistically, delayed uplift occurs when the trans secondary structures that cause the shift in signal did not for some reason immediately form as the second strand of a double stranded polynucleotide starts exiting the trans exit of pore, however, some period later part way through the transit of the second strand, the trans hybridization/secondary structure does occur and the signal shifts to “uplifted”. This is an example of the intra-strand inconsistent behavior.

Accuracy at both the single-molecule level and consensus pile-up level can be improved by increasing the extent and/or consistency of the formation of both the known structural elements (e.g. rehybridisation of the complementary strand of dsDNA/dsRNA) or random structural elements (sequence dependent regions that may form one or more than one type of structural element). Secondary structure affects nanopore discrimination and enzyme movement. Basecalling accuracy is fundamentally dependent on learning the underlying discrimination and movement behaviours, so if there are more varieties of behaviour to learn, the algorithm has to be more complex. As complexity and choices increase there is more chance of making an error. Alternatively, an algorithm may learn average behaviour, which does not perfectly describe the variety of true underlying behaviours, and is therefore inherently less accurate. Accuracy can be increased by reducing the absolute number of variable processes (random formation of expected or unexpected structural elements) or reducing the probability that alternative processes can form (e.g. ideally making one process dominant, most preferably above 50%, above 60%, above 70%, above 80%, above 90% or 100%). Increasing consistency may therefore result in the reduction of the number of different secondary structure types that can form, or increase the probability of occurrence of one type of process, or decrease the probability of occurrence of an alternative process.

The method of characterising a target polynucleotide may comprise: (a) providing a transmembrane pore in a membrane, wherein the membrane defines a first side and a second side, wherein the second side provides a condition that controls formation of a secondary structure from a portion of the target polynucleotide that moves through the transmembrane pore to the second side; (b) adding the target polynucleotide and a molecular brake to the first side, wherein the molecular brake binds to the target polynucleotide and controls the movement of the target polynucleotide through the pore; and (c) measuring one or more physical parameters that are indicative of one or more characteristics of the target polynucleotide as the target polynucleotide moves through the transmembrane pore.

### Random Secondary Structure

The selected conditions preferably control the extent and/or consistency of formation of random secondary structure by the target polynucleotide on the other side of the pore. The conditions may decrease, reduce or prevent the formation of random secondary structure. The conditions may increase the formation of random secondary structure. The formation of random secondary structure may be decreased, reduced, prevented or increased by any of the amounts discussed above.

Random secondary structures are secondary/tertiary structures in a polynucleotide that are in thermal equilibrium under the running conditions of the method. Random secondary structures can either be totally unformed, partially formed, or fully formed at any one time, and therefore at the moment of measurement as the polynucleotide passes through a transmembrane pore. Adjusting the conditions of the system as described herein will alter the balance between unformed and fully formed secondary/tertiary structures.

The random secondary structure preferably comprises (a) one or more helices, (b) one or more loops, (c) one or more pseudoknots, (d) one or more quadruplexes or (c) a combination thereof. In (e), the random secondary structure preferably comprises {a}; {b}; {c}; {d}; {a,b};{a,c}; {a,d}; {b,c}; {b,d}; {c,d}; {a,b,c}; {a,b,d}; {a,c,d}; {b,c,d}; or {a,b,c,d}. In any of (a) to (d), the random secondary structure may comprise any number of helices, loops, pseudoknots or quadruplexes, such as 2 or more, 3 or more, 4 or more, 5 or more, 10 or more, 20 or more, 50 or more, 100 or more, 500 or more, 1000 or more or 5000 or more.

The one or more helices in (a) may be any length. For example, the helices may comprise 2 or more, 3 or more, 4 or more, 5 or more, 10 or more, 20 or more, 50 or more, 100 or more or 500 or more nucleotides, and/or up to about 1000 or less, 500 or less, 100 or less, 50 or less, 10 or less or 5 or less nucleotides.

The one or more loops in (b) preferably comprise (i) one or more stem loops, (ii) one or more tetraloops, (iii) one or more hairpin loops or (iv) any combination thereof. In (iv), the one or more loops may comprise {i}; {ii}; {iii}; {i,ii}; {i,iii}, {ii, iii}; or {i,ii,iii,iv}. There may be any number of loops as discussed above. Stem loops are known in the art and may be any length. For example, the loops may comprise 2 or more, 3 or more, 4 or more, 5 or more, 10 or more, 20 or more, 50 or more, 100 or more or 500 or more nucleotides, and/or up to about 1000 or less, 500 or less, 100 or less, 50 or less, 10 or less or 5 or less nucleotides.

Hairpin loops are discussed in more detail below. Tetraloops are four-base hairpin loop motifs in RNA secondary structure that cap many double helices. There are many variants of the tetraloop, the published examples of which include ANYA, CUYG, GNRA, UMAC and UNCG.

The one or more pseudoknots in (c) may be any of those known in the art. A pseudoknot is a polynucleotide structure containing at least two stem loop structures in which half of one stem is intercalated between the two halves of another stem.

The one or more quadruplexes in (d) may be any of those known in the art. The one or more quadruplexes may be any type of quadruplex. The one or more quadruplexes may be one or more intermolecular quadruplexes, such as bimolecular quadruplexes or tetramolecular quadruplexes. The one or more quadruplexes are preferably intramolecular quadruplexes.

The one or more quadruplexes are preferably G-quadruplexes (also known as G-tetrads or G4-DNA). These are polynucleotide sequences that are rich in guanine and are capable of forming a four-stranded structure. Four guanine bases can associate through Hoogsteen hydrogen bonding to form a square planar structure called a guanine tetrad, and two or more guanine tetrads can stack on top of each other to form a G-quadruplex. The quadruplex structure is further stabilized by the presence of a cation, especially potassium, which sits in a central channel between each pair of tetrads. Forming G-quadruplexes is well known in the art (Marathias and Bolton, Nucleic Acids Research, 2000; 28 (9): 1969-1977; Kankia and Marky, J. Am. Chem. Soc. 2001, 123, 10799-10804; and Marusic et al., Nucleic Acids Research, 2012, 1-11). The one or more quadruplexes more preferably comprises the sequence Ga followed by Nb followed by Gc followed by Nd followed by Ge followed by Nf followed by Gg, wherein G is a nucleotide comprising guanine, wherein a, c, e and g are independently selected from 1, 2, 3, 4 and 5, wherein N is any nucleotide and wherein b, d and f are from 2 to 50. The values of a, c, e and g may be identical. G is preferably guanosine monophosphate (GMP), cyclic guanosine monophosphate (cGMP), deoxyguanosine monophosphate (dGMP), dideoxyguanosine monophosphate, N2-methyl-GMP, N2-methyl-cGMP, N2-methyl-dGMP, N2-methyl-dideoxyguanosine monophosphate, N2-methyl-06-methyl-GMP, N2-methyl-06-methyl-cGMP, N2-methyl-06-methyl-dGMP, N2-methyl-06-methyl-dideoxyguanosine monophosphate, 2′-O-methyl-GMP, 2′-O-methyl-cGMP, 2′-O-methyl-dGMP, 2′-O-methyl-dideoxyguanosine monophosphate, 6-thio-GMP, 6-thio-cGMP, 6-thio-dGMP, 6-thio-dideoxyguanosine monophosphate, 7-methyl-GMP, 7-methyl-cGMP, 7-methyl-dGMP, 7-methyl-dideoxyguanosine monophosphate, 7-deaza-GMP, 7-deaza-cGMP, 7-deaza-dGMP, 7-deaza-dideoxyguanosine monophosphate, 8-oxo-GMP, 8-oxo-cGMP, 8-oxo-dGMP or 8-oxo-dideoxyguanosine monophosphate.

The formation of random secondary structure on the other side of the pore can cause the polynucleotide to pull on the molecular brake and interfere with its function. The conditions may decrease, reduce or prevent the formation of random secondary structure on the other side of the pore and maintain the function of the molecular brake. The method preferably allows the molecular brake to display reduced forward slipping. This is a phenomenon where the polynucleotide moves forwards relative to the pore by at least 4 consecutive nucleotides and typically by more than 10 consecutive nucleotides. Slipping forward may involve movement forward of 100 consecutive nucleotides or more and this may happen more than once for each polynucleotide. Slipping forward can be problematic for polynucleotide sequencing.

The method preferably reduces the frequency of forward slipping displayed by the helicase by at least 10%, at least 20%, at least 30%, at least 40%, at least 50%, at least 60%, at least 70%, at least 80% or at least 90%. The reduction in frequency of slipping is typically compared to a reference condition. A typical reference condition is 150 mM potassium ferricyanide, 150 mM potassium ferrocyanide, 25 mM potassium phosphate, pH8 at 34° C. The method more preferably abolishes forward slipping, i.e. reduces the frequency of forward slipping displayed by the molecular brake by 100%. The method preferably reduces the length of forward slipping displayed by the molecular brake to 10 nucleotides or fewer, such as 9 nucleotides or fewer, 8 nucleotides or fewer, 7 nucleotides or fewer, 6 nucleotides or fewer, 5 nucleotides or fewer, 4 nucleotides or fewer, 3 nucleotides or fewer, 2 nucleotides or fewer, or 1 nucleotide. The method preferably reduces the frequency and length of forward slipping displayed by the molecular brake.

Forward slipping can be measured using any method known in the art. The ability of a molecular brake to control the movement of a polynucleotide and the incidence of forward slipping is typically assayed in a nanopore system, such as the ones described below.

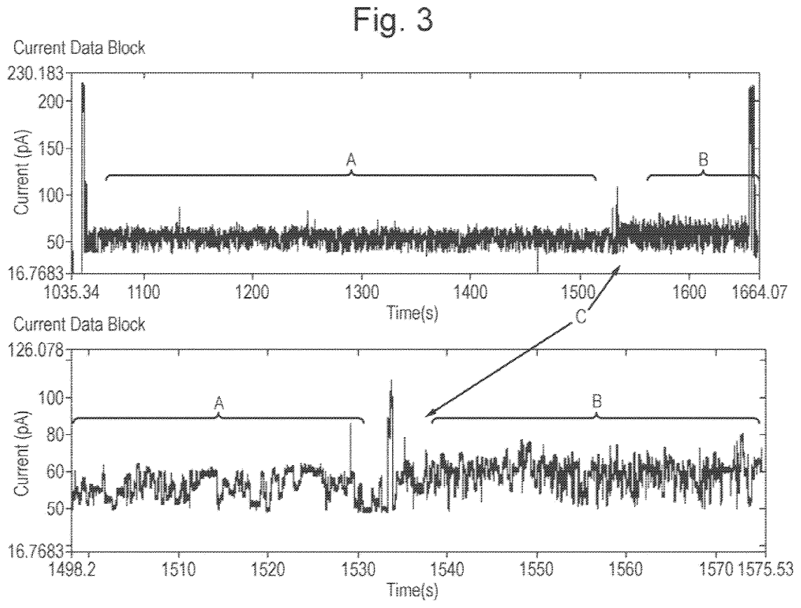

The method typically concerns reducing the formation of random secondary structure on the other side of the pore. This may reduce the phenomenon of uplift. Secondary structure formation on the other side of the pore, such as the formation of random secondary structure or the rehybrisation of the two strands of a double stranded target polynucleotide, applies a force to the polynucleotide, which is transmitted through to the sections of polynucleotide in the pore and through to the molecular brake. This may be observed by a change in the charaterisation measurements taken as the polynucleotide in the pore. With rehybrisation, the phenomenon typically manifests itself as an increase in current (typically average current) flowing through the pore when the complement sequence (the second strand) moves through the pore relative to the template sequence (first strand) and an increase in the characterisation current range. This can easily be seen by eye (see B versus A in FIG. 3). Hence, the phenomenon has been termed “uplift”. Uplift not only involves an increase in current range, but also typically involves an increased speed of movement of the complement sequence through the pore compared with the template sequence such that the duration of its characterisation is reduced. In addition to rehybridization, the formation of random secondary structure on the other side of the pore can also lead to uplift, albeit typically to a smaller degree and in a random manner. Uplift (or more specifically a shift in discrimination relative to ssDNA with no secondary structure passing through pore) is not limited to the complement. Uplift can occur in a target polynucleotide (e.g. DNA/RNA) if random structural elements form, for example; loops, hairpins, quadruplexes. Conversely, if uplift forms in a complement strand that is fully hybridising on the other side of the pore, there can still be “non-uplifted” regions due to structural elements (e.g. quadruplexes) that disrupt the perfect hybridisation on the other side of the pore.

Single stranded polynucleotides (e.g. DNA) with no ability to form secondary or tertiary structure is generally the baseline signal. The ability to form secondary or tertiary structure is not limited to two template-complement hybridized polynucleotides, it can form in single stranded DNA or single stranded RNA, e.g. Hairpin loops or quadruplexes. Therefore, as a template polynucleotide strand passes through a pore, secondary/tertiary structural elements can form that shift the signal. The formation of the structures can be inconsistent as they are stochastic, creating a random element to the signal that is difficult to model and can reduce basecalling accuracy. This occurs when sequencing two strands of a double stranded polynucleotide joined by a hairpin and can also occur when sequencing a single strand of a double stranded nucleotide.

In a case where uplift occurs when a complement strand exiting the pore is hybridizing to a template strand on the other side of the pore in an “ideal” manner (ic. Where there are no mismatches in the annealing), the signal can change back to “non-uplifted” if the “ideal” template-complement hybridization is disrupted, for example by other structural elements (e.g. a quadruplex structure) or by DNA damage.

Reduction of the formation of random secondary structure on the other side of the pore may therefore reduce random uplift. If the target polynucleotide is double stranded as discussed in more detail below, reducing the formation of random secondary structure on the other side of the pore may not necessarily reduce the uplift caused by rehybridisation. It may, however, make the rehybridisation of the two strands of the target polynucleotide on the other side of the pore more consistent and hence the uplift more consistent. Consistency facilitates the characterisation of the target polynucleotide because the uplift can be better predicted and accounted for.

The method may involve increasing the formation of random secondary structure on the other side of the pore. Although this may affect the random uplift caused by the formation of the random secondary structure itself, it may also to prevent the two strands of a double stranded target polynucleotide from rehybridising on the other side of the pore and thereby prevent the large uplift caused by such rehybridisation. This is discussed in more detail below. The changes in signal can be used to determine various characteristics of the polynucleotide, including but not limited to GC content, damage, modified bases and/or quadruplexes. If the behavior is random with <100% probability of formation, then combined information from a consensus pile-up of many separate strands/molecules can be used to determine the underlying properties. Increasing the chance of the signal change makes the features easier to measure and requires less coverage in a consensus pile-up.

### Rehybridisation

In preferred embodiments, the target polynucleotide is double stranded. In such embodiments, the method preferably comprises providing a construct comprising the target polynucleotide in which the two strands of the target polynucleotide are linked at one end of the target polynucleotide by a hairpin loop. The method more preferably comprises the step of linking the two strands of the target polynucleotide at one end of the target polynucleotide by a hairpin loop to provide a construct comprising the target polynucleotide. These steps are discussed in more detail below. The construct is contacted with one side of a transmembrane pore in a membrane and a molecular brake which separates the two strands of the construct and controls the movement of the construct through the pore one strand at a time. One or more measurements are taken which are indicative of one or more characteristics of the target polynucleotide as the construct moves with respect to the pore.

Suitable hairpin loops can be designed using methods known in the art. Hairpin loops typically comprise a stem region formed from the hybridisation of two parts of the hairpin loop and a loop region. The loop region typically does not comprise any hybridisation. The hairpin loop may be any length. The hairpin loop is typically 110 or fewer nucleotides, such as 100 or fewer nucleotides, 90 or fewer nucleotides, 80 or fewer nucleotides, 70 or fewer nucleotides, 60 or fewer nucleotides, 50 or fewer nucleotides, 40 or fewer nucleotides, 30 or fewer nucleotides, 20 or fewer nucleotides or 10 or fewer nucleotides, in length. The hairpin loop is preferably from about 1 to 110, from 2 to 100, from 5 to 80 or from 6 to 50 nucleotides in length. Longer lengths of the hairpin loop, such as from 50 to 110 nucleotides, are preferred if the loop is involved in the differential selectability of the adaptor. Similarly, shorter lengths of the hairpin loop, such as from 1 to 5 nucleotides, are preferred if the loop is not involved in the selectable binding as discussed below. The stem region may be any length. Typically, the stem region has a length of from about 4 to about 10 bases up to about 100, 1000, 10000, 100000 or 1000000 bases.

The hairpin loop may be provided at either end of the polynucleotide, i.e. 5′ or 3′ end. The hairpin loop may be ligated to the polynucleotide using any method known in the art. The hairpin loop may be ligated using a ligase, such as T4 DNA ligase, E. coli DNA ligase, Taq DNA ligase, Tma DNA ligase and 9°N DNA ligase.

The two strands of the polynucleotide may be separated using any method known in the art. For instance, they may be separated by a molecular brake, such as a polynucleotide binding protein, or using conditions which favour dehybridsation (examples of conditions which favour dehybridisation include, but are not limited to, high temperature, high pH and the addition of agents that can disrupt hydrogen bonding or base pairing, such as formamide and urea). The hairpin loop preferably comprises a selectable binding moiety. This allows the polynucleotide to be purified or isolated. A selectable binding moiety is a moiety that can be selected on the basis of its binding properties. Hence, a selectable binding moiety is preferably a moiety that specifically binds to a surface. A selectable binding moiety specifically binds to a surface if it binds to the surface to a much greater degree than any other moiety used in the invention. In preferred embodiments, the moiety binds to a surface to which no other moiety used in the invention binds.

Suitable selective binding moieties are known in the art. Preferred selective binding moieties include, but are not limited to, biotin, a polynucleotide sequence, antibodies, antibody fragments, such as Fab and ScSv, antigens, polynucleotide binding proteins, poly histidine tails and GST tags. The most preferred selective binding moieties are biotin and a selectable polynucleotide sequence. Biotin specifically binds to a surface coated with avidins. Selectable polynucleotide sequences specifically bind (i.e. hybridise) to a surface coated with homologus sequences. Alternatively, selectable polynucleotide sequences specifically bind to a surface coated with polynucleotide binding proteins.

The hairpin loop and/or the selectable binding moiety may comprise a region that can be cut, nicked, cleaved or hydrolysed. This allows the hairpin loop to be selectively cleaved on the other side of the pore as discussed in more detail below. Such a region can be designed to allow the polynucleotide to be removed from the surface to which it is bound following purification or isolation. Suitable regions are known in the art. Suitable regions include, but are not limited to, an RNA region, a region comprising desthiobiotin and streptavidin, a disulphide bond and a photocleavable region.

The conditions on the other side of the pore control the formation of secondary structure by the target polynucleotide on the other side of the pore. The conditions preferably control the ability of the two strands of the target polynucleotide to rehybridise on the other side of the pore. The conditions preferably control the formation of random secondary structure by the target polynucleotide on the other side of the pore. The conditions preferably control the ability of the two strands of the target polynucleotide to rehybridise on the other side of the pore and control the formation of random secondary structure by the target polynucleotide on the other side of the pore. The conditions more preferably reduce the ability of the two strands of the target polynucleotide to rehybridise on the other side of the pore by increasing the formation of random secondary structure by the target polynucleotide on the other side of the pore. The control of the formation of random secondary structure is discussed above.

The conditions may decrease, reduce or prevent the ability of the two strands of the target polynucleotide to rehybridise on the other side of the pore. The conditions may prevent the two strands of the target polynucleotide from rehybridising on the other side of the pore. The method may involve increasing the formation of random secondary structure on the other side of the pore to prevent or stop the strands of a double stranded target polynucleotide from rehybridising on the other side of the pore. This is advantageous because it reduces or abolishes the phenomenon of uplift as discussed above.

The conditions may increase the ability of the two strands of the target polynucleotide to rehybridise on the other side of the pore. The rehybridisation may be decreased, reduced, prevented or increased by any of the amounts discussed above. Increasing the rehybridisation may make uplift more consistent or predictable.

Methods of Controlling Secondary Structure and/or Rehybridisation

The conditions preferably comprise one or more species on the other side of the pore which control or decrease the formation of secondary structure by the target polynucleotide. The one or more species may control or decrease the formation of random secondary structure as described above. The one or more species may control or decrease the ability of the two strands of the target polynucleotide to rehybridise.

The one or more species preferably comprise (i) one or more control polynucleotides which hybridise with the target polynucleotide and/or the hairpin loop. The conditions preferably comprise (i) a plurality of control polynucleotides which hybridise with the target polynucleotide and/or the hairpin loop. The conditions may comprise any number of control polynucleotides, such as 2, 5, 10, 20, 50, 100, 500, 1000, 5000 or 10,000 or more. The one or more controlled polynucleotides may be formed from any of the polynucleotides discussed below.

The one or more control polynucleotides preferably comprise universal nucleotides. A universal nucleotide is one which will hybridise to some degree to all of the nucleotides in the target polynucleotide. A universal nucleotide is preferably one which will hybridise to some degree to nucleotides comprising the nucleosides adenosine (A), thymine (T), uracil (U), guanine (G) and cytosine (C). The universal nucleotide may hybridise more strongly to some nucleotides than to others. For instance, a universal nucleotide inosine (I) comprising the nucleoside, 2′-deoxyinosine, will show a preferential order of pairing of I-C>I-A>I-G approximately =I-T. For the purposes of the invention, it is only necessary that the universal nucleotide used in the one or more control polynucleotides hybridises to all of the nucleotides in the target polynucleotide.

The universal nucleotide preferably comprises one of the following nucleobases: hypoxanthine, 4-nitroindole, 5-nitroindole, 6-nitroindole, 3-nitropyrrole, nitroimidazole, 4-nitropyrazole, 4-nitrobenzimidazole, 5-nitroindazole, 4-aminobenzimidazole or phenyl(C6-aromatic ring. The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside or phenyl C-2′-deoxyribosyl nucleoside. The universal nucleotide most preferably comprises 2′-deoxyinosine.

The one or more control polynucleotides preferably comprise pseudo-complementary nucleotides. Pseudo-complementary (PC) nucleotides contain base analogs that form weak base pairs with one another, but form strong base pairs with standard bases. The strength of base pairing can be determining by standard means in the art, such as by measuring the melting temperature. Consequently, PC-based polynucleotides have diminished intramolecular and intermolecular secondary structures and can readily hybridize to unmodified polynucleotides. One example of a pseudo-complementary base pair is the one between 2-aminoadenine (nA) and 2-thiothymine (sT). Compared with the Watson-Crick base pair between adenine (A) and thymine (T), the pair between nA and sT is unstable because of the steric clash between the exocyclic amine of nA and the large size of the sulphur atom of ST. While the nA: sT base pair is unstable, the base pairing strength of the A: sT pair is similar to that of an A:T base pair. The nA:T pair is more stable than A:T, presumably because three hydrogen bonds can now be formed between nA and T. The use of PC nucleotides in the one or more control polynucleotides means that the control nucleotides will hybridize with the target polynucleotide on the other side of the pore but will not hybridise with each other.

The one or more control polynucleotides may be complementary to the target polynucleotide and/or the hairpin loop. The hairpin loop is added to the target polynucleotide and so its sequence is known. It is therefore possible to design one or more control polynucleotides which are complementary to the hairpin loop, hybridise to the hairpin loop on the other side of the pore and prevent rehybridisation. The sequence of the target polynucleotide is typically unknown. However, it is straightforward to design a population of control polynucleotides which contain every combination of nucleotides which can be used to hybridise to the target polynucleotide. For instance, if the control polynucleotides are 4 nucleotides in length and A, G, T and C nucleotides (see below) are used to create them, a population containing 4⁴ (256) control polynucleotides can be designed which contain every possible combination of A, G, T and C. Any members of this population whose complementary sequences appear in the unknown sequence of the target sequence will hybridise to the unknown target sequence on the other side of the pore. The control polynucleotide is preferably sufficiently complementary to the target polynucleotide that it binds to the target polynucleotide under assay conditions for 0.5 ms or more, such as for from 1 ms to 1 s or 1 ms to 10 ms. The one or more control polynucleotides may be any length. For instance, they may be at least 4, at least 5, at least 10, at least 15 or at least 20 nucleotides in length. Such lengths are suitable when the one or more control polynucleotides comprise universal nucleotides. The one or more control polynucleotides are preferably at least 30, at least 60 or at least 90 nucleotides in length. The one or more control polynucleotides may be up to 100, 200, 500, 1000, 10000 or 100000 nucleotides in length. The control strand may be longer than, the same length as or shorter than the target polynucleotide.

The one or more control polynucleotides preferably comprise one or more anchors which are capable of coupling the one or more control polynucleotides to the membrane. Suitable anchors are discussed below with reference to the target polynucleotide.

The one or more species preferably comprise (ii) one or more proteins or one or more chemicals which bind to the target polynucleotide and/or the hairpin loop. The conditions preferably comprise (ii) a plurality of proteins or chemicals which bind to the target polynucleotide and/or the hairpin loop. The conditions may comprise any number of proteins or chemicals, such as 2, 5, 10, 20, 50, 100, 500, 1000, 5000 or 10,000 or more. The one or more proteins may be any proteins which bind to single stranded polynucleotides. The proteins may be any of the polynucleotide binding proteins discussed below. The one or more proteins are preferably one or more single-stranded binding proteins (SSBs) or are one or more helicases. If the target polynucleotide is RNA, the one or more proteins may be one or more ribozymes.

The one or more chemicals may be any of the chemicals discussed below with reference to molecular brakes, such as one or more cyclodextrins.

The one or more species preferably comprise (iii) a nuclease. The nuclease is preferably an endonuclease, an exonuclease or a uracil-specific excision reagent (USERs). The nuclease digests the polynucleotide on the other side of the pore and prevents the formation of secondary structure and/or prevents rehybridisation.

The one or more species may comprise a combination of (i), (ii) and (iii), such as (i), (ii), (iii), (i) and (ii), (ii) and (iii), (i) and (iii) or (i), (ii) and (iii).

The species may be any molecule that can bind to the polynucleotide after it has passed through the transmembrane pore to inhibit secondary structure formation. There are many known types of proteins that bind to DNA/RNA. Such proteins include motor proteins but are preferably passive binders, for example, SSB, T4 gp32 or recA proteins. The species may be a molecular brake but does not not need to bind so strongly that it can slow a polynucleotide moving through a nanopore under force. The species only has to block or slow the formation of secondary/tertiary structure. Chemical species include, for example, intercalators, of which many are known in the art.

The conditions preferably comprise (a) a higher salt concentration on the other side of the pore than on the one side of the pore and/or (b) a lower pH on the other side of the pore than on the one side of the pore. The salt is preferably potassium glutamate (K-glutamate) or potassium chloride (KCl). In one embodiment, a higher salt concentration is typically a salt concentration that is about 5% or more or 10% or more greater. In another embodiment, a higher salt concentration is typically a difference of 10 mM or more, such as 100 mM or more up to about 1M, 2M or 3M. In one embodiment, a lower pH is a pH of 0.5 pH units or more less.

The conditions preferably chemically or physically cleave the target polynucleotide on the other side of the pore. The conditions preferably comprise contacting the target polynucleotide on the other side of the pore with one or more restriction enzymes. The conditions preferably comprise passing the target polynucleotide through a small hole on the other side of the pore under pressure. The pressure could, by way of example, be voltage difference, osmotic pressure or fluidic pressure. A pressure differential can be applied across membranes/nanopores. The pressure may be a physical pressure applied by physical means (e.g. using a pump or syringe), or an osmotic pressure arising as a result of a difference in chemical composition on the different sides of the membrane. The conditions preferably comprise nebulising the target polynucleotide on the other side of the pore. Other suitable conditions include, but are not limited to, the following:
Restriction digestion.
Acoustic shearing by the transmission of high-frequency acoustic energy waves.
Nebulization by forcing the polynucleotide through a small hole in a nebulizer unit.
Sonication.
Point-sink shearing, a type of hydrodynamic shearing, uses a syringe pump to create hydrodynamic shear forces by polynucleotides through a small abrupt contraction.
Needle shearing by passing the polynucleotides through small gauge needle.
French pressure cells pass polynucleotides through a narrow valve under high pressure to create high shearing forces.
Transposome mediated fragmentation (tagmentation).

The conditions preferably comprise chemically cleaving the target polynucleotide on the other side of the pore. The target polynucleotide is preferably chemically cleaved using one or more of Fe(II)-ethylenediaminetetraacetic acid (Fe(II)-EDTA), piperidine, benzenediazonium tetrafluoroborate, a metallo-intercalator and a metallo-inserter.

The conditions preferably comprise selectively cleaving the hairpin loop on the other side of the pore. The hairpin loop is preferably selectively cleaved by an enzyme. The loop may be designed so that it is selectively targeted by a restriction enzyme or a different type of nuclease, such as Cas9. The loop may be designed such that it self cleaves. For instance, certain ribozyme structures self cleave.

The conditions preferably comprise one or more species on the other side of the pore which increase the formation of secondary structure by the target polynucleotide. The one or more species are preferably interchelators or metal cofactors. Suitable metal cofactors include, but are not limited to, magnesium, such as Mg2+, magnesium (II) chloride or magnesium (II) acetate; cobalt, such as Co2+, cobalt (II) chloride or cobalt (II) acetate; manganese, such as Mn2+, manganese (II) chloride or manganese (II) acetate; zinc, such as Zn2+, zinc (II) sulphate monohydrate; calcium, such as Ca2+, calcium (II) chloride or calcium (II) acetate monohydrate; aluminium, such as A13+ or aluminium (III) chloride; beryllium, such as Be2+ or beryllium (II) sulphate; and nickel, such as Ni2+ or nickel (II) sulphate hexhydrate. The metal cofactor is preferably potassium, such as K+ or potassium chloride.

### Hairpin Design

The invention provides an improved method of characterising a double stranded target polynucleotide. The target polynucleotide is provided as part of a construct in which the two strands of the target polynucleotide are linked at one end of the target polynucleotide by a hairpin loop. The method more preferably comprises the step of linking the two strands of the target polynucleotide at one end of the target polynucleotide by a hairpin loop to provide a construct comprising the target polynucleotide. The construct is contacted with one side of a transmembrane pore in a membrane. The construct is also contacted with a molecular brake which separates the two strands of the construct and controls the movement of the construct through the pore one strand at a time. Suitable pores, membranes and molecular brakes are discussed in more detail below. One or more measurements which are indicative of one or more characteristics of the target polynucleotide are taken as the construct moves with respect to the pore. An important part of the invention is that the hairpin loop is designed to control the ability of the two strands of the target polynucleotide to rehybridise on the other side of the pore. In other words, the hairpin is designed to control the rehybridisation of the two strands of the target polynucleotide/construct after it has moved through the pore.

The ability of the two strands of the target polynucleotide to rehybridise on the other side of the pore may be controlled in any way and to any extent. The hairpin loop may be designed to decrease, reduce or prevent the rehybridisation. The hairpin loop may decrease, reduce or prevent the rehybridisation by any amount, such by at least 10%, at least 20%, at least 30%, at least 40%, at least 50%, at least 60%, at least 70%, at least 80% or at least 90%. The hairpin more preferably abolishes the rehybridisation (i.e. decreases, reduces or prevents it by 100%).

Alternatively, the hairpin may be designed to increase the rehybridisation. The hairpin may increase the rehybridisation by any amount, such by at least 10%, at least 20%, at least 30%, at least 40%, at least 50%, at least 60%, at least 70%, at least 80% or at least 90%. The hairpin may increase the rehybridisation by at least twofold, at least threefold, at least fourfold, at least fivefold, at least tenfold, at least twentyfold, at least thirtyfold, at least fortyfold, at least fiftyfold, at least one hundredfold, at least five hundredfold or one thousandfold.

Rehybridisation may be measured in any known way. For example, rehybridisation may be measured by melting.

The hairpin loop is preferably designed to decrease the ability of the two strands of the target polynucleotide to rehybridise on the other side of the pore. The hairpin loop is more preferably designed to prevent the two strands of the target polynucleotide from rehybridising on the other side of the pore.

Preferably, the hairpin loop (a) can be selectively cleaved on the other side of the pore, (b) comprises a loop region having a length of 60 or more nucleotides, (c) comprises one or more sequences capable of forming random secondary structure, (d) has a reduced a melting temperature (Tm) or (c) any combination thereof. In (e), the hairpin preferably {a}; {b}; {c};{d}; {a,b}; {a,c}; {a,d}; {b,c}; {b,d}; {c,d}; {a,b,c}; {a,b,d}; {a,c,d}; {b,c,d}; or {a,b,c,d}. The length of the hairoin loop may be from about 60 to about 1000 nucleotides such at about 100 to about 200 nucleotides.

The structure of hairpin loops is discussed above with reference to suitable “non-designed” hairpin loops for use in the invention.

In (a), selective cleavage of the hairpin loops means that the two strands of the target polynucleotide may move apart and reduce the likelihood of rehybridisation. Selective cleavage of hairpin loops is discussed above.

In (b), the loop region may be any length. The loop region may have a length of 70 or more, 80 or more, 90 or more, 100 or more, 125 or more or 150 or more nucleotides. The loop may comprise any of the nucleotides discussed below. The loop may comprise a long stretch of polyT (a long stretch of only nucleotides containing thymine (T)). Such streteches may be 25 or more, 50 or more or 75 or more nucleotides in length. The loop may comprise one or more abasic nucleotides. A longer loop reduces the likelihood that the two strands of the target polynucleotide will rehybridise on the other side of the pore.

In (c), the hairpin loop may comprise one or more sequences capable of forming random secondary structure. The hairpin loop may comprise any number of sequences capable of forming random secondary structure, such as two or more, three or more, five or more or ten or more. The random secondary structure may be any of those discussed above. The hairpin loop preferably comprises one or more sequences which are capable of forming (a) one or more helices, (b) one or more loops, (c) one or more pseudoknots, (d) one or more quadruplexes or (e)a combination thereof, such as {a}; {b}; {c}; {d}; {a,b}; {a,c}; {a,d}; {b,c}; {b,d}; {c,d};{a,b,c}; {a,b,d}; {a,c,d}; {b,c,d}; or {a,b,c,d}. The hairpin loop preferably comprises one or more sequences which are capable of forming one or more quadruplexes. The hairpin loop preferably comprises one or more quadruplexes with additional duplex structures, such as HD22 (a psudeo G4 with additional duplex structure). The sequence of HD22 is shown in the Examples. Such sequences are discussed above. The formation of random secondary structure by the hairpin loop on the other side of the pore reduces the likelihood that the two strands of the target polynucleotide will rehybridise on the other side of the pore.

In (d), the hairpin may have any reduction in melting temperature (Tm). Melting temperature and how it may be measured is discussed below. The stem region of the hairpin loop may comprise one or more mismatches, such as 1, 2, 3, 4, 5, 6, 7, 8 or more mismatches. Melting temperature may also be reduced by including one or more, such as 1, 2, 3, 4, 5, 6, 7, 8 or more universal nucleotides. Universal nucleotides are discussed in more detail below.

The hairpin loop is preferably designed to decrease the ability of the two strands of the target polynucleotide to rehybridise on the other side of the pore. However, the hairpin loop must be able to form a loop before it is ligated to and used to link the two strands of the target polynucleotide. This can be achieved by forming the loop in the absence of free metal cofactors, such as in the absence of free potassium ions. The metal cofactors may be any of those discussed above. Such cofactors may be sequestered (such that they are not free) using a chelator, such as EDTA (Ethylenediaminetetraacetic acid). The presence of the cofactors on the other side of the pore allows the designed hairpin to function in accordance with the invention.

The hairpin loop is preferably designed to increase the ability of the two strands of the target polynucleotide to rehybridise on the other side of the pore. This allows the uplift to be more consistent and predictable.

Preferably, the hairpin loop (a) comprises a loop region having a length of 20 or fewer nucleotides, such as 10 or fewer or 5 or fewer nucleotides and/or (b) has an increased melting temperature (Tm). Melting temperature may be increased by forming the stem region from nucleotides which hybridise to each other more strongly than natural DNA or RNA nucleotides, such as BNA nucleotides.

### Polynucleotide

The target polynucleotide may be any polynucleotide. The control polynucleotide may also be any type of polynucleotide.

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag. Suitable labels are described below. The polynucleotide may comprise one or more spacers.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose.

The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The polynucleotide can comprise one strand of RNA hybridised to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety. Bridged nucleic acids (BNAs) are modified RNA nucleotides. They are sometimes also referred to as constrained or inaccessible RNA molecules. BNA monomers can contain a five-membered, six-membered or even a seven-membered bridged structure with a “fixed” C3′-endo sugar puckering. The bridge is synthetically incorporated at the 2′, 4′-position of the ribose to afford a 2′, 4′-BNA monomer. The monomers can be incorporated into oligonucleotide polymeric structures using standard phosphoamidite chemistry. BNAs are structurally rigid oligo-nucleotides with increased binding affinities and stability.

The polynucleotide is most preferably ribonucleic nucleic acid (RNA) or deoxyribonucleic acid (DNA).

The target polynucleotide can be any length. For example, the target polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

The control polynucleotide can be any length. For example, the control polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. In one embodiment, the control polynucleotide is shorter than the target polynucleotide, by from about 2 to 500000 nucleotides, such as about 10 to 500, 50 to 300, or 100 to 200 nucleotides shorter.

### Sample

The target polynucleotide may be present in any suitable sample. The sample may be abiological sample. The invention may be carried out in vitro using at least one sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on at least one sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep, fish, chickens or pigs or may alternatively be pets such as cats or dogs. Alternatively, the sample may be of plant origin, such as a sample obtained from a commercial crop, such as a cereal, legume, fruit or vegetable, for example wheat, barley, oats, canola, maize, soya, rice, rhubarb, bananas, apples, tomatoes, potatoes, grapes, tobacco, beans, lentils, sugar cane, cocoa, cotton.

The sample may be a non-biological sample. The non-biological sample is preferably a fluid sample. Examples of non-biological samples include surgical fluids, water such as drinking water, sea water or river water, and reagents for laboratory tests.

The sample is typically processed prior to being used in the invention, for example by centrifugation or by passage through a membrane that filters out unwanted molecules or cells, such as red blood cells. The sample may be measured immediately upon being taken. The sample may also be typically stored prior to assay, preferably below −70° C.

### Membrane

The target polynucleotide is contacted with one side of a transmembrane pore in a membrane. Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units are polymerised together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

The membrane is most preferably one of the membranes disclosed in International Application No. PCT/GB2013/052766 (published as WO 2014/064443) or PCT/GB2013/052767 (published as WO 2014/064444).

The amphiphilic molecules may be chemically-modified or functionalised to facilitate coupling of the polynucleotide.

The amphiphilic layer may be a monolayer or a bilayer. The amphiphilic layer is typically planar. The amphiphilic layer may be curved. The amphiphilic layer may be supported. The amphiphilic layer may be concave. The amphiphilic layer may be suspended from raised such that the peripheral region of the amphiphilic layer (which is attached to the pillars) is higher than the amphiphilic layer region. This may allow the microparticle to travel, move, slide or roll along the membrane as described above.

Amphiphilic membranes are typically naturally mobile, essentially acting as two dimensional fluids with lipid diffusion rates of approximately 10-8 cm s-1. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. PCT/GB08/004127 (published as WO 2009/077734). Advantageously in this method, the lipid bilayer is formed from dried lipids. In a most preferred embodiment, the lipid bilayer is formed across an opening as described in WO2009/077734 (PCT/GB08/004127).

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such as surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy (Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane is a solid state layer. Solid state layers can be formed from both organic and inorganic materials including, but not limited to, microelectronic materials, insulating materials such as HfO₂, Si₃N₄, Al₂O₃, and SiO, organic and inorganic polymers such as polyamide, plastics such as TEFLON® or elastomers such as two-component addition-cure silicone rubber, and glasses. The solid state layer may be by atomic layer deposition (ALD). The ALD solid state layer may comprise alternating layers of HfO₂ and Al₂O₃. The solid state layer may be formed from monatomic layers, such as graphene, or layers that are only a few atoms thick. Suitable graphene layers are disclosed in International Application No. PCT/US2008/010637 (published as WO 2009/035647). Yusko et al., Nature Nanotechnology, 2011; 6:253-260 and US Patent Application No. 2013/0048499 describe the delivery of proteins to transmembrane pores in solid state layers without the use of microparticles. The method of the invention may be used to improve the delivery in the methods disclosed in these documents.

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Transmembrane Pore

A transmembrane pore is a structure that crosses the membrane to some degree. Typically, a transmembrane pore comprises a first opening and a second opening with a lumen extending between the first opening and the second opening. The transmembrane pore permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any transmembrane pore may be used in the invention. The pore may be biological or artificial. Suitable pores include, but are not limited to, protein pores, polynucleotide pores and solid state pores. The pore may be a DNA origami pore (Langecker et al., Science, 2012; 338:932-936).

The transmembrane pore is preferably a transmembrane protein pore. A transmembrane protein pore is a polypeptide or a collection of polypeptides that permits hydrated ions, such as polynucleotide, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide, such as DNA or RNA, to be moved through the pore.

The transmembrane protein pore may be a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with s, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F(OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from lysenin. Suitable pores derived from CsgG are disclosed in International Application No. PCT/EP2015/069965. Suitable pores derived from lysenin are disclosed in International Application No. PCT/GB2013/050667 (published as WO 2013/153359). The transmembrane pore may be derived from or based on Msp, α-hemolysin (α-HL), lysenin, CsgG, ClyA, Sp1 and haemolytic protein fragaceatoxin C(FraC). The wild type α-hemolysin pore is formed of 7 identical monomers or sub-units (i.e., it is heptameric). The sequence of one monomer or sub-unit of α-hemolysin-NN is shown in SEQ ID NO: 4.

The transmembrane protein pore is preferably derived from Msp, more preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. Preferably the pore is derived from MspA or a homolog or paralog thereof.

A monomer derived from Msp typically comprises the sequence shown in SEQ ID NO: 2 or a variant thereof. SEQ ID NO: 2 is the MS-(B1) 8 mutant of the MspA monomer. It includes the following mutations: D90N, D91N, D93N, D118R, D134R and E139K. A variant of SEQ ID NO: 2 is a polypeptide that has an amino acid sequence which varies from that of SEQ ID NO: 2 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for inserting subunits into membranes, such as amphiphilic layers. For example, subunits may be suspended in a purified form in a solution containing a triblock copolymer membrane such that it diffuses to the membrane and is inserted by binding to the membrane and assembling into a functional state. Alternatively, subunits may be directly inserted into the membrane using the “pick and place” method described in M.A. Holden, H. Bayley. J. Am. Chem. Soc. 2005, 127, 6502-6503 and International Application No. PCT/GB2006/001057 (published as WO 2006/100484).

Over the entire length of the amino acid sequence of SEQ ID NO: 2, a variant will preferably be at least 50% homologous to that sequence based on amino acid similarity or identity. More preferably, the variant may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 2 over the entire sequence. There may be at least 80%, for example at least 85%, 90% or 95%, amino acid similarity or identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300;Altschul, S. F et al (1990) J Mol Biol 215:403-10. Software for performing BLAST analyses is publicly available through the National Center for Biotechnology Information (http://www.ncbi.nlm.nih.gov/). Similarity can be measured using pairwise identity or by applying a scoring matrix such as BLOSUM62 and converting to an equivalent identity. Since they represent functional rather than evolved changes, deliberately mutated positions would be masked when determining homology. Similarity may be determined more sensitively by the application of position-specific scoring matrices using, for example, PSIBLAST on a comprehensive database of protein sequences. A different scoring matrix could be used that reflect amino acid chemico-physical properties rather than frequency of substitution over evolutionary time scales (e.g. charge).

SEQ ID NO: 2 is the MS-(B1) 8 mutant of the MspA monomer. The variant may comprise any of the mutations in the MspB, C or D monomers compared with MspA. The mature forms of MspB, C and D are shown in SEQ ID NOs: 5 to 7. In particular, the variant may comprise the following substitution present in MspB:A138P. The variant may comprise one or more of the following substitutions present in MspC:A96G, N102E and A138P. The variant may comprise one or more of the following mutations present in MspD:Deletion of G1,L2V, E5Q, L8V, D13G, W21A, D22E, K47T, 149H, 168V, D91G, A96Q, N102D, S103T, V1041, S136K and G141A. The variant may comprise combinations of one or more of the mutations and substitutions from Msp B, C and D. The variant preferably comprises the mutation L88N. A variant of SEQ ID NO: 2 has the mutation L88N in addition to all the mutations of MS-B1 and is called MS-(B2)8. The pore used in the invention is preferably MS-(B2)8. The variant of SEQ ID NO: 2 preferably comprises one or more of D56N, D56F, E59R, G75S, G77S, A96D and Q126R. A variant of SEQ ID NO: 2 has the mutations G75S/G77S/L88N/Q126R in addition to all the mutations of MS-B1 and is called MS-B2C. The pore used in the invention is preferably MS-(B2) 8 or MS-(B2C) 8. The variant of SEQ ID NO: 2 preferably comprises N93D. The variant more preferably comprises the mutations G75S/G77S/L88N/N93D/Q126R.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid.

The transmembrane protein pore is preferably derived from CsgG, more preferably from CsgG from E. coli Str. K-12 substr. MC4100. Such a pore will be oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others.

A monomer derived from CsgG typically comprises the sequence shown in SEQ ID NO: 27 or a variant thereof. A variant of SEQ ID NO: 27 is a polypeptide that has an amino acid sequence which varies from that of SEQ ID NO: 27 and which retains its ability to form a pore. The ability of a variant to form a pore can be assayed using any method known in the art as discussed above.

Over the entire length of the amino acid sequence of any one of SEQ ID NO: 27, a variant will preferably be at least 50% homologous to that sequence based on amino acid similarity or identity. More preferably, the variant may be at least 55%, at least 60%, at least 65%, at least 70%, at least 75%, at least 80%, at least 85%, at least 90% and more preferably at least 95%, 97% or 99% homologous based on amino acid similarity or identity to the amino acid sequence of SEQ ID NO: 27 over the entire sequence. There may be at least 80%, for example at least 85%, 90% or 95%, amino acid similarity or identity over a stretch of 100 or more, for example 125, 150, 175 or 200 or more, contiguous amino acids (“hard homology”). Homology can be measured as discussed above.

The variant of SEQ ID NO: 27 may comprise any of the mutations disclosed in International Application No. PCT/GB2015/069965 (published as WO 2016/034591). The variant of SEQ ID NO: 27 preferably comprises one or more of the following (i) one or more mutations at the following positions (i.e. mutations at one or more of the following positions) N40, D43, E44, S54, S57, Q62, R97, E101, E124, E131, R142, T150 and R192, such as one or more mutations at the following positions (i.e. mutations at one or more of the following positions) N40, D43, E44, S54, S57, Q62, E101, E131 and T150 or N40, D43, E44, E101 and E131; (ii) mutations at Y51/N55, Y51/F56, N55/F56 or Y51/N55/F56; (iii) Q42R or Q42K; (iv) K49R; (v) N102R, N102F, N102Y or N102W; (vi) D149N, D149Q or D149R; (vii) E185N, E185Q or E185R; (viii) D195N, D195Q or D195R; (ix) E201N, E201Q or E201R; (x) E203N, E203Q or E203R; and (xi) deletion of one or more of the following positions F48, K49, P50,Y51, P52, A53, S54, N55, F56 and S57. The variant may comprise any combination of (i) to (xi). If the variant comprises any one of (i) and (iii) to (xi), it may further comprise a mutation at one or more of Y51, N55 and F56, such as at Y51, N55, F56, Y51/N55, Y51/F56, N55/F56 or Y51/N55/F56.

Preferred variants of SEQ ID NO: 27 which form pores in which fewer nucleotides contribute to the current as the polynucleotide moves through the pore comprise Y51A/F56A, Y51A/F56N, Y511/F56A, Y51L/F56A, Y51T/F56A, Y511/F56N, Y51L/F56N or Y51T/F56N or more preferably Y511/F56A, Y51L/F56A or Y51T/F56A.

Preferred variants of SEQ ID NO: 27 which form pores displaying an increased range comprise mutations at the following positions:
Y51, F56, D149, E185, E201 and E203;
N55 and F56;
Y51 and F56;
Y51, N55 and F56; or
F56 and N102.

Preferred variants of SEQ ID NO: 27 which form pores displaying an increased range comprise:
Y51N, F56A, D149N, E185R, E201N and E203N;
N55S and F56Q;
Y51A and F56A;
Y51A and F56N;
Y511 and F56A;
Y51L and F56A;
Y51T and F56A;
Y511 and F56N;
Y51L and F56N;
Y51T and F56N;
Y51T and F56Q;
Y51A, N55S and F56A;
Y51A, N55S and F56N;
Y51T, N55S and F56Q; or
F56Q and N102R.

Preferred variants of SEQ ID NO: 27 which form pores in which fewer nucleotides contribute to the current as the polynucleotide moves through the pore comprise mutations at the following positions:
N55 and F56, such as N55X and F56Q, wherein X is any amino acid; or
Y51 and F56, such as Y51X and F56Q, wherein X is any amino acid.

Preferred variants of SEQ IDNO: 27 which form pores displaying an increased throughput comprise mutations at the following positions:
D149, E185 and E203;
D149, E185, E201 and E203; or
D149, E185, D195, E201 and E203.

Preferred variants which form pores displaying an increased throughput comprise:
D149N, E185N and E203N;
D149N, E185N, E201N and E203N;
D149N, E185R, D195N, E201N and E203N; or
D149N, E185R, D195N, E201R and E203N.

Preferred variants of SEQ ID NO: 7 which form pores in which capture of the polynucleotide is increased comprise the following mutations:
D43N/Y51T/F56Q;
E44N/Y51T/F56Q;
D43N/E44N/Y51T/F56Q;
Y51T/F56Q/Q62R;
D43N/Y51T/F56Q/Q62R;
E44N/Y51T/F56Q/Q62R; or
D43N/E44N/Y51T/F56Q/Q62R.

Preferred variants of SEQ ID NO: 27 comprise the following mutations:
D149R/E185R/E201R/E203R or Y51T/F56Q/D149R/E185R/E201R/E203R;
D149N/E185N/E201N/E203N or Y51T/F56Q/D149N/E185N/E201N/E203N;
E201R/E203R or Y51T/F56Q/E201R/E203R
E201N/E203R or Y51T/F56Q/E201N/E203R;
E203R or Y51T/F56Q/E203R;
E203N or Y51T/F56Q/E203N;
E201R or Y51T/F56Q/E201R;
E201N or Y51T/F56Q/E201N;
E185R or Y51T/F56Q/E185R;
E185N or Y51T/F56Q/E185N;
D149R or Y51T/F56Q/D149R;
D149N or Y51T/F56Q/D149N;
R142E or Y51T/F56Q/R142E;
R142N or Y51T/F56Q/R142N;
R192E or Y51T/F56Q/R192E; or
R192N or Y51T/F56Q/R192N.

Preferred variants of SEQ ID NO: 27 comprise the following mutations:
Y51A/F56Q/E101N/N102R;
Y51A/F56Q/R97N/N102G;
Y51A/F56Q/R97N/N102R;
Y51A/F56Q/R97N;
Y51A/F56Q/R97G;
Y51A/F56Q/R97L;
Y51A/F56Q/N102R;
Y51A/F56Q/N102F;
Y51A/F56Q/N102G;
Y51A/F56Q/E101R;
Y51A/F56Q/E101F;
Y51A/F56Q/E101N; or
Y51A/F56Q/E101G

The variant of SEQ ID NO: 27 may comprise any of the substitutions present in another CsgG homologue. Preferred CsgG homologues are shown in SEQ ID NOs: 3 to 7 and 26 to 41 of International Application No. PCT/GB2015/069965 (published as WO 2016/034591).

Any of the proteins described herein, such as the transmembrane protein pores, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the pore or construct. An example of this would be to react a gel-shift reagent to a cysteine engineered on the outside of the pore. This has been demonstrated as a method for separating hemolysin hetero-oligomers (Chem Biol. 1997 July;4 (7): 497-505).

The pore may be labelled with a revealing label. The revealing label may be any suitable label which allows the pore to be detected. Suitable labels include, but are not limited to, fluorescent molecules, radioisotopes, e.g. ¹²⁵I, ³⁵S, enzymes, antibodies, antigens, polynucleotides and ligands such as biotin.

Any of the proteins described herein, such as the transmembrane protein pores, may be made synthetically or by recombinant means. For example, the pore may be synthesised by in vitro translation and transcription (IVTT). The amino acid sequence of the pore may be modified to include non-naturally occurring amino acids or to increase the stability of the protein. When a protein is produced by synthetic means, such amino acids may be introduced during production. The pore may also be altered following either synthetic or recombinant production.

Any of the proteins described herein, such as the transmembrane protein pores, can be produced using standard methods known in the art. Polynucleotide sequences encoding a pore or construct may be derived and replicated using standard methods in the art. Polynucleotide sequences encoding a pore or construct may be expressed in a bacterial host cell using standard techniques in the art. The pore may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The pore may be produced in large scale following purification by any protein liquid chromatography system from protein producing organisms or after recombinant expression. Typical protein liquid chromatography systems include FPLC, AKTA™ systems, the Bio-Cad system, the Bio-Rad BIOLOGIC™ system and the Gilson HPLC system.

### Microparticle

A microparticle may be used to deliver the target polynucleotide to the transmembrane pore. Any number of microparticles can be used in the method of the invention. For instance, the method of the invention may use a single microparticle or 2, 3, 4, 5, 6, 7, 8, 9, 10, 20, 30, 50, 100, 1,000, 5,000, 10,000, 100,000, 500,000 or 1,000,000 or more microparticles. If two or more microparticles are used, the microparticles may be the same. Alternatively, a mixture of different microparticles may be used.

Each microparticle may have one polynucleotide attached. Alternatively, each microparticle may have two or more polynucleotides, such as 3 or more, 4 or more, 5 or more, 6 or more, 7 or more, 8 or more, 9 or more, 10 or more, 20 or more, 30 or more, 50 or more, 100 or more, 500 or more, 1,000 or more, 5,000 or more, 10,000 or more, 100,000 or more, 1000,000 or more or 5000,000 or more polynucleotides, attached. A microparticle may be substantially or completed coated or covered with polynucleotide. A microparticle may have a polynucleotide attached over substantially all of or all of its surface. A microparticle may be attached to a polynucleotide via an adaptor. The adaptor may be a Y-adaptor or a hairpin adaptor (see below)

A polynucleotide, i.e. a single instance of an polynucleotide, may be attached to two or more microparticles. A polynucleotide, i.e. a single instance of an polynucleotide, may be attached to any number of the microparticles discussed above.

A microparticle is a microscopic particle whose size is typically measured in micrometres (μm). Microparticles may also known as microspheres or microbeads. The microparticle may be a nanoparticle. A nanoparticle is a microscopic particle whose size is typically measured in nanometres (nm).

A microparticle typically has a particle size of from about 0.001 μm to about 500 μm. For instance, a nanoparticle may have a particle size of from about 0.01 μm to about 200 μm or about 0.1 μm to about 100 μm. More often, a microparticle has a particle size of from about 0.5 μm to about 100 μm, or for instance from about 1 μm to about 50 μm. The microparticle may have a particle size of from about 1 nm to about 1000 nm, such as from about 10 nm to about 500 nm, about 20 nm to about 200 nm or from about 30 nm to about 100 nm.

A microparticle may be spherical or non-spherical. Spherical microparticles may be called microspheres. Non-spherical particles may for instance be plate-shaped, needle-shaped, irregular or tubular. The term “particle size” as used herein means the diameter of the particle if the particle is spherical or, if the particle is non-spherical, the volume-based particle size. The volume-based particle size is the diameter of the sphere that has the same volume as the non-spherical particle in question.

If two or more microparticles are used in the method, the average particle size of the microparticles may be any of the sizes discussed above, such as from about 0.5 μm to about 500 μm. A population of two or more microparticles preferably has a coefficient of variation (ratio of the standard deviation to the mean) of 10% or less, such as 5% or less or 2% or less.

Any method may be used to determine the size of the microparticle. Suitable methods include, but are not limited to, flow cytometry (see, for example, Chandler et al., J Thromb Haemost. 2011 June;9 (6): 1216-24).

The microparticle may be formed from any material. The microparticle is preferably formed from a ceramic, glass, silica, a polymer or a metal. The polymer may be a natural polymer, such as polyhydroxyalkanoate, dextran, polylactide, agarose, cellulose, starch or chitosan, or a synthetic polymer, such as polyurethane, polystyrene, poly(vinyl chloride), silane or methacrylate. Suitable microparticles are known in the art and are commercially available. Ceramic and glass microspheres are commercially available from 3M®. Silica and polymer microparticles are commercially available from EPRUI Nanoparticles & Microspheres Co. Ltd. Microparticles are also commercially available from Polysciences Inc., Bangs Laboratories Inc. and Life Technologies.

The microparticle may be solid. The microparticle may be hollow. The microparticle may be formed from polymer fibers.

The microparticle may be derived from the kit used to extract and isolate the polynucleotide.

The surface of the microparticle may interact with and attach the polynucleotide. The surface may naturally interact with the polynucleotide without functionalisation. The surface of the microparticle is typically functionalised to facilitate attachment of the polynucleotide. Suitable functionalisations are known in the art. For instance, the surface of the microparticle may be functionalised with a polyhistidine-tag (hexa histidine-tag, 6xHIS-TAG®, HIS6 TAG® or HIS-TAG®), Ni-NTA, streptavidin, biotin, an oligonucleotide, a polynucleotide (such as DNA, RNA, PNA, GNA, TNA or LNA), carboxyl groups, quaternary amine groups, thiol groups, azide groups, alkyne groups, DIBO, lipid, FLAG-tag (FLAG octapeptide, polynucleotide binding proteins (including any of those discussed below), peptides, proteins, antibodies or antibody fragments. Antibody fragments are discussed in more detail below. The microparticle may also be functionalised with any of the linkers or groups discussed below with reference to attachment.

The microparticle may be functionalised with a molecule or group which specifically binds to the polynucleotide. In this instance, the polynucleotide which will be attached to the microparticle and delivered to the transmembrane pore may be called the target polynucleotide. This allows the microparticle to select or capture the target polynucleotide from a sample containing other polynucleotides. A molecule or group specifically binds to the target polynucleotide if it binds to the target polynucleotide with preferential or high affinity, but does not bind or binds with only low affinity to other or different polynucleotides. A molecule or group binds with preferential or high affinity if it binds with a Kd of 1×10⁶ M or less, more preferably 1×10⁻⁷ M or less, 5×10⁻⁸ M or less, more preferably 1×10⁻⁸ M or less or more preferably 5×10⁻⁹ M or less. A molecule or group binds with low affinity if it binds with a Kd of 1×10⁻⁶ M or more, more preferably 1×10⁵ M or more, more preferably 1×10⁻⁴ M or more, more preferably 1×10⁻³ M or more, even more preferably 1×10⁻² M or more.

Preferably, the molecule or group binds to the target polynucleotide with an affinity that is at least 10 times, such as at least 50, at least 100, at least 200, at least 300, at least 400, at least 500, at least 1000 or at least 10,000 times, greater than its affinity for other polynucleotides. Affinity can be measured using known binding assays, such as those that make use of fluorescence and radioisotopes. Competitive binding assays are also known in the art. The strength of binding between peptides or proteins and polynucleotides can be measured using nanopore force spectroscopy as described in Hornblower et al., Nature Methods. 4:315-317.(2007).

The microparticle may be functionalised with an oligonucleotide or a polynucleotide (such as any of those discussed above) which specifically hybridises to the target polynucleotide or comprises a portion or region which is complementary to a portion or region of the target polynucleotide. This allows the microparticle to select or capture the target polynucleotide from a sample containing other polynucleotides. An oligonucleotide or polynucleotide specifically hybridises to a target polynucleotide when it hybridises with preferential or high affinity to the target polynucleotide but does not substantially hybridise, does not hybridise or hybridises with only low affinity to other polynucleotide. An oligonucleotide or polynucleotide specifically hybridises if it hybridises to the target polynucleotide with a melting temperature (Tm) that is at least 2° C., such as at least 3° C., at least 4° C., at least 5° C., at least 6° C., at least 7° C., at least 8° C., at least 9° C. or at least 10° C., greater than its Tm for other sequences. More preferably, the oligonucleotide or polynucleotide hybridises to the target polynucleotide with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for other nucleic acids. Preferably, the oligonucleotide or polynucleotide hybridises to the target polynucleotide with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for a sequence which differs from the target polynucleotide by one or more nucleotides, such as by 1, 2, 3, 4 or 5 or more nucleotides. The oligonucleotide or polynucleotide typically hybridises to the target polynucleotide with a Tm of at least 90° C., such as at least 92° C. or at least 95° C. Tm can be measured experimentally using known techniques, including the use of DNA microarrays, or can be calculated using publicly available Tm calculators, such as those available over the internet.

Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1× (0.1650 M Na⁺) to 2× (0.33 M Na⁺) SSC (standard sodium citrate) at 50° C.

Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na⁺) to 1× (0.1650 M Na⁺) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na⁺) SSC at 60° C.

The oligonucleotide or polynucleotide may comprise a portion or region which is substantially complementary to a portion or region of the target polynucleotide. The region or portion of the oligonucleotide or polynucleotide may therefore have 1, 2, 3, 4, 5, 6, 7, 8, 9, 10 or more mismatches across a region of 5, 10, 15, 20, 21, 22, 30, 40 or 50 nucleotides compared with the portion or region in the target polynucleotide.

A portion of region is typically 50 nucleotides or fewer, such as 40 nucleotides or fewer, 30 nucleotides or fewer, 20 nucleotides or fewer, 10 nucleotides or fewer or 5 nucleotides or fewer.

The microparticle is preferably paramagnetic or magnetic. The microparticle preferably comprises a paramagnetic or a superparamagnetic material or a paramagnetic or a superparamagnetic metal, such as iron. Any suitable magnetic microparticle may be used. For instance, magnetic beads commercially available from, for instance, Clontech, Promega, Invitrogen ThermoFisher Scientific and NEB, may be used. In some embodiments, the microparticle comprises a magnetic particle with an organic group such as a metal-chelating group, such as nitrilotriacetic acid (NTA), attached. The organic component may, for instance, comprise a group selected from —C(═O)O—, —C—O—C—, —C(═O)—, —NH—, —C(═O)—NH, —C(═O)—CH₂—I, —S(═O)₂— and —S—. The organic component may comprise a metal chelating group, such as NTA (nitrilotriacetic acid). Usually, a metal such as gold, iron, nickel or cobalt is also attached to the metal-chelating group. Magnetic beads of this sort are commonly used for capturing His-tagged proteins, but are also suitable for use in the invention.

The microparticle is most preferably a HIS-TAG® DYNABEAD® which is commercially available from Life Technologies, Mag Strep beads from IBA, Streptavidin magnetic beads from NEB, Solid Phase Reversible Immobilization (SPRI®) beads or Agencourt AMPure XP beads from Beckman Coulter or DYNADEADS® MYONE™ Streptavidin C1 (ThermoFisher Scientific).

### Coupling

The target polynucleotide preferably comprises one or more anchors which are capable of coupling to the membrane. The method preferably further comprises coupling the target polynucleotide to the membrane using the one or more anchors.

The anchor comprises a group which couples (or binds) to the polynucleotide and a group which couples (or binds) to the membrane. Each anchor may covalently couple (or bind) to the polynucleotide and/or the membrane.

The polynucleotide may be coupled to the membrane using any number of anchors, such as 2, 3, 4 or more anchors. For instance, the polynucleotide may be coupled to the membrane using two anchors each of which separately couples (or binds) to both the polynucleotide and membrane.

The one or more anchors may comprise one or more molecular brakes. Each anchor may comprise one or more molecular brakes. The molecular brake(s) may be any of those discussed below.

If the membrane is an amphiphilic layer, such as a triblock copolymer membrane, the one or more anchors preferably comprise a polypeptide anchor present in the membrane and/or a hydrophobic anchor present in the membrane. The hydrophobic anchor is preferably a lipid, fatty acid, sterol, carbon nanotube, polypeptide, protein or amino acid, for example cholesterol, palmitate or tocopherol. In preferred embodiments, the one or more anchors are not the pore.

The components of the membrane, such as the amphiphilic molecules, copolymer or lipids, may be chemically-modified or functionalised to form the one or more anchors.

Examples of suitable chemical modifications and suitable ways of functionalising the components of the membrane are discussed in more detail below. Any proportion of the membrane components may be functionalised, for example at least 0.01%, at least 0.1%, at least 1%, at least 10%, at least 25%, at least 50% or 100%.

The polynucleotide may be coupled directly to the membrane. The one or more anchors used to couple the polynucleotide to the membrane preferably comprise a linker. The one or more anchors may comprise one or more, such as 2, 3, 4 or more, linkers. One linker may be used to couple more than one, such as 2, 3, 4 or more, polynucleotides to the membrane.

Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The polynucleotide may hybridise to a complementary sequence on the circular polynucleotide linker.

The one or more anchors or one or more linkers may comprise a component that can be cut or broken down, such as a restriction site or a photolabile group.

Functionalised linkers and the ways in which they can couple molecules are known in the art. For instance, linkers functionalised with maleimide groups will react with and attach to cysteine residues in proteins. In the context of this invention, the protein may be present in the membrane, may be the polynucleotide itself or may be used to couple (or bind) to the polynucleotide. This is discussed in more detail below.

Crosslinkage of polynucleotides can be avoided using a “lock and key” arrangement. Only one end of each linker may react together to form a longer linker and the other ends of the linker each react with the polynucleotide or membrane respectively. Such linkers are described in International Application No. PCT/GB10/000132 (published as WO 2010/086602).

The use of a linker is preferred in the sequencing embodiments discussed below. If a polynucleotide is permanently coupled directly to the membrane in the sense that it does not uncouple when interacting with the pore, then some sequence data will be lost as the sequencing run cannot continue to the end of the polynucleotide due to the distance between the membrane and the pore. If a linker is used, then the polynucleotide can be processed to completion.

The coupling may be permanent or stable. In other words, the coupling may be such that the polynucleotide remains coupled to the membrane when interacting with the pore.

The coupling may be transient. In other words, the coupling may be such that the polynucleotide may decouple from the membrane when interacting with the pore. For certain applications, such as aptamer detection and polynucleotide sequencing, the transient nature of the coupling is preferred. If a permanent or stable linker is attached directly to either 5′ or 3′ end of a polynucleotide and the linker is shorter than the distance between the membrane and the transmembrane pore's channel, then some sequence data will be lost as the sequencing run cannot continue to the end of the polynucleotide. If the coupling is transient, then when the coupled end randomly becomes free of the membrane, then the polynucleotide can be processed to completion. Chemical groups that form permanent/stable or transient links are discussed in more detail below. The polynucleotide may be transiently coupled to an amphiphilic layer or triblock copolymer membrane using cholesterol or a fatty acyl chain. Any fatty acyl chain having a length of from 6 to 30 carbon atom, such as hexadecanoic acid, may be used.

In preferred embodiments, a polynucleotide, such as a nucleic acid, is coupled to an amphiphilic layer such as a triblock copolymer membrane or lipid bilayer. Coupling of nucleic acids to synthetic lipid bilayers has been carried out previously with various different tethering strategies. These are summarised in Table 3 below.

TABLE 3

| Anchor | Type of | |
|---|---|---|
| comprising | coupling | Reference |
| Thiol | Stable | Yoshina-Ishii, C. and S. G. Boxer (2003). |
| | | “Arrays of mobile tethered vesicles on |
| | | supported lipid bilayers.” J Am Chem Soc |
| | | 125(13): 3696-7. |
| Biotin | Stable | Nikolov, V., R. Lipowsky, et al. (2007). |
| | | “Behavior of giant vesicles with anchored D |
| | | NA molecules.” Biophys J 92(12): 4356-68 |
| Cholesterol | Transient | Pfeiffer, I. and F. Hook (2004). “Bivalent |
| | | cholesterol-based coupling of oligonucletides |
| | | to lipid membrane assemblies.” |
| | | J Am Chem Soc 126(33): 10224-5 |
| Surfactant | Stable | van Lengerich, B., R. J. Rawle, et al. |
| (e.g. Lipid, | | “Covalent attachment of lipid vesicles to |
| Palmitate, | | a fluid-supported bilayer allows observation |
| etc) | | of DNA-mediated vesicle interactions.” |
| | | Langmuir 26(11): 8666-72 |

Synthetic polynucleotides and/or linkers may be functionalised using a modified phosphoramidite in the synthesis reaction, which is easily compatible for the direct addition of suitable anchoring groups, such as cholesterol, tocopherol, palmitate, thiol, lipid and biotin groups. These different attachment chemistries give a suite of options for attachment to polynucleotides. Each different modification group couples the polynucleotide in a slightly different way and coupling is not always permanent so giving different dwell times for the polynucleotide to the membrane. The advantages of transient coupling are discussed above.

Coupling of polynucleotides to a linker or to a functionalised membrane can also be achieved by a number of other means provided that a complementary reactive group or an anchoring group can be added to the polynucleotide. The addition of reactive groups to either end of a polynucleotide has been reported previously. A thiol group can be added to the 5′ of ssDNA or dsDNA using T4 polynucleotide kinase and ATPγS (Grant, G. P. and P. Z. Qin (2007). “A facile method for attaching nitroxide spin labels at the 5′ terminus of nucleic acids.” Nucleic Acids Res 35 (10): e77). An azide group can be added to 5′-phosphate of ssDNA or dsDNA using T4 polynucleotide kinase and γ-[2-Azidoethyl]-ATP or γ-[6-Azidohexyl]-ATP. Using thiol or Click chemistry a tether, containing either a thiol, iodoacetamide OPSS or maleimide group (reactive to thiols) or a DIBO (dibenzocyclooxtyne) or alkyne group (reactive to azides), can be covalently attached to the polynucleotide. A more diverse selection of chemical groups, such as biotin, thiols and fluorophores, can be added using terminal transferase to incorporate modified oligonucleotides to 3′ of ssDNA (Kumar, A., P. Tchen, et al. (1988). “Nonradioactive labeling of synthetic oligonucleotide probes with terminal deoxynucleotidyl transferase.” Anal Biochem 169 (2): 376-82). Streptavidin/biotin and/or streptavidin/desthiobiotin coupling may be used for any other polynucleotide. The Examples below describes how a polynucleotide can be coupled to a membrane using streptavidin/biotin and streptavidin/desthiobiotin. It may also be possible that anchors may be directly added to polynucleotides using terminal transferase with suitably modified nucleotides (e.g. cholesterol or palmitate).

The one or more anchors preferably couple the polynucleotide to the membrane via hybridisation. The hybridisation may be present in any part of the one or more anchors, such as between the one or more anchors and the polynucleotide, within the one or more anchors or between the one or more anchors and the membrane. Hybridisation in the one or more anchors allows coupling in a transient manner as discussed above. For instance, a linker may comprise two or more polynucleotides, such as 3, 4 or 5 polynucleotides, hybridised together. The one or more anchors may hybridise to the polynucleotide. The one or more anchors may hybridise directly to the polynucleotide, directly to a Y adaptor and/or leader sequence attached to the polynucleotide or directly to a hairpin loop adaptor attached to the polynucleotide (as discussed in more detail below). Alternatively, the one or more anchors may be hybridised to one or more, such as 2 or 3, intermediate polynucleotides (or “splints”) which are hybridised to the polynucleotide, to a Y adaptor and/or leader sequence attached to the polynucleotide or to a hairpin loop adaptor attached to the polynucleotide (as discussed in more detail below).

The one or more anchors may comprise a single stranded or double stranded polynucleotide. One part of the anchor may be ligated to a single stranded or double stranded polynucleotide analyte. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89 (20): 9823-5). Alternatively, either a single stranded or double stranded polynucleotide can be ligated to a double stranded polynucleotide and then the two strands separated by thermal or chemical denaturation. To a double stranded polynucleotide, it is possible to add either a piece of single stranded polynucleotide to one or both of the ends of the duplex, or a double stranded polynucleotide to one or both ends. For addition of single stranded polynucleotides to the double stranded polynucleotide, this can be achieved using T4 RNA ligase I as for ligation to other regions of single stranded polynucleotides. For addition of double stranded polynucleotides to a double stranded polynucleotide then ligation can be “blunt-ended”, with complementary 3′ dA/dT tails on the polynucleotide and added polynucleotide respectively (as is routinely done for many sample prep applications to prevent concatemer or dimer formation) or using “sticky-ends” generated by restriction digestion of the polynucleotide and ligation of compatible adapters. Then, when the duplex is melted, each single strand will have either a 5′ or 3′ modification if a single stranded polynucleotide was used for ligation or a modification at the 5′ end, 3′ end or both if a double stranded polynucleotide was used for ligation.

If the polynucleotide is a synthetic strand, the one or more anchors can be incorporated during the chemical synthesis of the polynucleotide. For instance, the polynucleotide can be synthesised using a primer having a reactive group attached to it.

Adenylated polynucleotides are intermediates in ligation reactions, where an adenosine-monophosphate is attached to 5′-phosphate of the polynucleotide. Various kits are available for generation of this intermediate, such as 5′ DNA Adenylation Kit from NEB. By substituting ATP in the reaction for a modified nucleotide triphosphate, then addition of reactive groups (such as thiols, amines, biotin, azides, etc) to 5′ of a polynucleotide can be possible. It may also be possible that anchors could be directly added to polynucleotides using a 5′ DNA adenylation kit with suitably modified nucleotides (e.g. cholesterol or palmitate).

A common technique for the amplification of sections of genomic DNA is using polymerase chain reaction (PCR). Here, using two synthetic oligonucleotide primers, a number of copies of the same section of DNA can be generated, where for each copy 5′ of each strand in the duplex will be a synthetic polynucleotide. Single or multiple nucleotides can be added to 3′ end of single or double stranded DNA by employing a polymerase. Examples of polymerases which could be used include, but are not limited to, Terminal Transferase, Klenow and E. coli Poly(A) polymerase). By substituting ATP in the reaction for a modified nucleotide triphosphate then anchors, such as cholesterol, thiol, amine, azide, biotin or lipid, can be incorporated into double stranded polynucleotides. Therefore, each copy of the amplified polynucleotide will contain an anchor.

Ideally, the polynucleotide is coupled to the membrane without having to functionalise the polynucleotide. This can be achieved by coupling the one or more anchors, such as a polynucleotide binding protein or a chemical group, to the membrane and allowing the one or more anchors to interact with the polynucleotide or by functionalizing the membrane. The one or more anchors may be coupled to the membrane by any of the methods described herein. In particular, the one or more anchors may comprise one or more linkers, such as maleimide functionalised linkers.

In this embodiment, the polynucleotide is typically RNA, DNA, PNA, TNA or LNA and may be double or single stranded. This embodiment is particularly suited to genomic DNA polynucleotides.

The one or more anchors can comprise any group that couples to, binds to or interacts with single or double stranded polynucleotides, specific nucleotide sequences within the polynucleotide or patterns of modified nucleotides within the polynucleotide, or any other ligand that is present on the polynucleotide.

Suitable binding proteins for use in anchors include, but are not limited to, E. coli single stranded binding protein, P5 single stranded binding protein, T4 gp32 single stranded binding protein, the TOPO V dsDNA binding region, human histone proteins, E. coli HU DNA binding protein and other archacal, prokaryotic or eukaryotic single stranded or double stranded polynucleotide (or nucleic acid) binding proteins, including those listed below.

The specific nucleotide sequences could be sequences recognised by transcription factors, ribosomes, endonucleases, topoisomerases or replication initiation factors. The patterns of modified nucleotides could be patterns of methylation or damage.

The one or more anchors can comprise any group which couples to, binds to, intercalates with or interacts with a polynucleotide. The group may intercalate or interact with the polynucleotide via electrostatic, hydrogen bonding or Van der Waals interactions. Such groups include a lysine monomer, poly-lysine (which will interact with ssDNA or dsDNA), ethidium bromide (which will intercalate with dsDNA), universal bases or universal nucleotides (which can hybridise with any polynucleotide) and osmium complexes (which can react to methylated bases). A polynucleotide may therefore be coupled to the membrane using one or more universal nucleotides attached to the membrane. Each universal nucleotide may be coupled to the membrane using one or more linkers. The universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl(C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-O′-methylinosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. The universal nucleotide more preferably comprises 2′-deoxyinosine. The universal nucleotide is more preferably IMP or dIMP. The universal nucleotide is most preferably dPMP (2′-Deoxy-P-nucleoside monophosphate) or dKMP (N6-methoxy-2, 6-diaminopurine monophosphate).

The one or more anchors may couple to (or bind to) the polynucleotide via Hoogsteen hydrogen bonds (where two nucleobases are held together by hydrogen bonds) or reversed Hoogsteen hydrogen bonds (where one nucleobase is rotated through 180° with respect to the other nucleobase). For instance, the one or more anchors may comprise one or more nucleotides, one or more oligonucleotides or one or more polynucleotides which form Hoogsteen hydrogen bonds or reversed Hoogsteen hydrogen bonds with the polynucleotide. These types of hydrogen bonds allow a third polynucleotide strand to wind around a double stranded helix and form a triplex. The one or more anchors may couple to (or bind to) a double stranded polynucleotide by forming a triplex with the double stranded duplex.

In this embodiment at least 1%, at least 10%, at least 25%, at least 50% or 100% of the membrane components may be functionalised.

Where the one or more anchors comprise a protein, they may be able to anchor directly into the membrane without further functonalisation, for example if it already has an external hydrophobic region which is compatible with the membrane. Examples of such proteins include, but are not limited to, transmembrane proteins, intramembrane proteins and membrane proteins. Alternatively the protein may be expressed with a genetically fused hydrophobic region which is compatible with the membrane. Such hydrophobic protein regions are known in the art.

The one or more anchors are preferably mixed with the polynucleotide before delivery to the membrane, but the one or more anchors may be contacted with the membrane and subsequently contacted with the polynucleotide.

In another aspect the polynucleotide may be functionalised, using methods described above, so that it can be recognised by a specific binding group. Specifically the polynucleotide may be functionalised with a ligand such as biotin (for binding to streptavidin), amylose (for binding to maltose binding protein or a fusion protein), Ni-NTA (for binding to poly-histidine or poly-histidine tagged proteins) or peptides (such as an antigen).

According to a preferred embodiment, the one or more anchors may be used to couple a polynucleotide to the membrane when the polynucleotide is attached to a leader sequence which preferentially threads into the pore. Leader sequences are discussed in more detail below. Preferably, the polynucleotide is attached (such as ligated) to a leader sequence which preferentially threads into the pore. Such a leader sequence may comprise a homopolymeric polynucleotide or an abasic region. The leader sequence is typically designed to hybridise to the one or more anchors either directly or via one or more intermediate polynucleotides (or splints). In such instances, the one or more anchors typically comprise a polynucleotide sequence which is complementary to a sequence in the leader sequence or a sequence in the one or more intermediate polynucleotides (or splints). In such instances, the one or more splints typically comprise a polynucleotide sequence which is complementary to a sequence in the leader sequence.

Any of the methods discussed above for coupling polynucleotides to membranes, such as amphiphilic layers, can of course be applied to other polynucleotide and membrane combinations. In some embodiments, an amino acid, peptide, polypeptide or protein is coupled to an amphiphilic layer, such as a triblock copolymer layer or lipid bilayer. Various methodologies for the chemical attachment of such polynucleotides are available. An example of a molecule used in chemical attachment is EDC (1-ethyl-3-[3-dimethylaminopropyl]carbodiimide hydrochloride). Reactive groups can also be added to the 5′ of polynucleotides using commercially available kits (Thermo Pierce, Part No. 22980). Suitable methods include, but are not limited to, transient affinity attachment using histidine residues and Ni-NTA, as well as more robust covalent attachment by reactive cysteines, lysines or non natural amino acids.

### Polynucleotide Characterisation

The method of the invention involves characterising a target polynucleotide. After the target polynucleotide is contacted with the pore, one or more measurements which are indicative of one or more characteristics of the target polynucleotide are taken as the polynucleotide moves with respect to the pore.

Any number of polynucleotides can be investigated. For instance, the method of the invention may concern characterising two or more polynucleotides, such as 3 or more, 4 or more, 5 or more, 6 or more, 7 or more, 8 or more, 9 or more, 10 or more, 20 or more, 30 or more, 50 or more, 100 or more, 500 or more, 1,000 or more, 5,000 or more, 10,000 or more, 100,000 or more, 1000,000 or more or 5000,000 or more, polynucleotides. The two or more polynucleotides may be delivered using the same microparticle or different microparticles.

If two or more polynucleotides are characterised, they may be different from one another. The two or more polynucleotides may be two or more instances of the same polynucleotide. This allows proof reading.

The polynucleotides can be naturally occurring or artificial. For instance, the method may be used to verify the sequence of two or more manufactured oligonucleotides. The methods are typically carried out in vitro.

The method may involve measuring two, three, four or five or more characteristics of each polynucleotide. The one or more characteristics are preferably selected from (i) the length of the polynucleotide, (ii) the identity of the polynucleotide, (iii) the sequence of the polynucleotide, (iv) the secondary structure of the polynucleotide and (v) whether or not the polynucleotide is modified. Any combination of (i) to (v) may be measured in accordance with the invention, such as {i}, {ii}, {iii}, {iv}, {v}, {i,ii}, {i,iii}, {i,iv}, {i,v}, {ii, iii}, {ii,iv}, {ii, v},{iii, iv}, {iii,v}, {iv,v}, {i,ii, iii}, {i,ii,iv}, {i,ii,v}, {i,iii, iv}, {i,iii,v}, {i,iv,v}, {ii,iii,iv}, {ii,iii,v},{ii,iv,v}, {iii,iv,v}, {i,ii, iii,iv}, {i,ii,iii,v}, {i,ii,iv,v}, {i,iii,iv,v}, {ii,iii,iv,v} or {i,ii,iii,iv,v}.

For (i), the length of the polynucleotide may be measured for example by determining the number of interactions between the polynucleotide and the pore or the duration of interaction between the polynucleotide and the pore.

For (ii), the identity of the polynucleotide may be measured in a number of ways. The identity of the polynucleotide may be measured in conjunction with measurement of the sequence of the polynucleotide or without measurement of the sequence of the polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12;106 (19): 7702-7, Lieberman K R et al, J Am Chem Soc. 2010; 132 (50): 17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the polynucleotide is modified by methylation, by oxidation, by damage, with one or more proteins or with one or more labels, tags or spacers. Specific modifications will result in specific interactions with the pore which can be measured using the methods described below. For instance, methylcyotsine may be distinguished from cytosine on the basis of the current flowing through the pore during its interaction with each nucleotide.

The methods may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The methods may be carried out using the apparatus described in International Application No. PCT/GB08/000562 (WO 2008/102120).

A variety of different types of measurements may be made. This includes without limitation: electrical measurements and optical measurements. A suitable optical method involving the measurement of fluorescence is disclosed by J. Am. Chem. Soc. 2009, 131 1652-1653. Possible electrical measurements include: current measurements, impedance measurements, tunnelling measurements (Ivanov A P et al., Nano Lett. 2011 Jan. 12; 11 (1): 279-85), and FET measurements (International Application WO 2005/124888). Optical measurements may be combined with electrical measurements (Soni G V et al., Rev Sci Instrum. 2010 January; 81 (1): 014301). The measurement may be a transmembrane current measurement such as measurement of ionic current flowing through the pore.

Electrical measurements may be made using standard single channel recording equipment as describe in Stoddart D et al., Proc Natl Acad Sci, 12;106 (19): 7702-7, Lieberman K R et al, J Am Chem Soc. 2010; 132 (50): 17961-72, and International Application WO 2000/28312. Alternatively, electrical measurements may be made using a multi-channel system, for example as described in International Application WO 2009/077734 and International Application WO 2011/067559.

The method is preferably carried out with a potential applied across the membrane. The applied potential may be a voltage potential. Alternatively, the applied potential may be a chemical potential. An example of this is using a salt gradient across a membrane, such as an amphiphilic layer. A salt gradient is disclosed in Holden et al., J Am Chem Soc. 2007 Jul. 11; 129 (27): 8650-5. In some instances, the current passing through the pore as a polynucleotide moves with respect to the pore is used to estimate or determine the sequence of the polynucleotide. This is strand sequencing.

The methods may involve measuring the current passing through the pore as the polynucleotide moves with respect to the pore. Therefore the apparatus may also comprise an electrical circuit capable of applying a potential and measuring an electrical signal across the membrane and pore. The methods may be carried out using a patch clamp or a voltage clamp. The methods preferably involve the use of a voltage clamp.

The methods of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

Molecular brake

The method comprises contacting the target polynucleotide with a molecular brake which controls the movement of the target polynucleotide through the pore. Any molecular brake may be used including any of those disclosed in International Application No. PCT/GB2014/052737 (published as WO 2015/110777).

The molecular brake is preferably a polynucleotide binding protein. The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The polynucleotide binding protein preferably is able to slip, slide or actively move along the polynucleotide when under force (e.g. from an applied electrical field) and in contact with a nanopore. The polynucleotide binding protein is preferably one that does not immediately disengage from the polynucleotide when brought into contact with a nanopore. The polynucleotide binding protein is preferably not covalently joined to the polynucleotide, or otherwise locked to the polynucleotide in a manner that prevents the polynucleotide moving relative to the polynucleotide and through a nanopore in contact with the polynucleotide binding protein.

The polynucleotide binding protein is preferably derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the polynucleotide and controlling its movement through the pore. For instance, the enzyme may be modified to remove its enzymatic activity or may be used under conditions which prevent it from acting as an enzyme. Such conditions are discussed in more detail below.

The polynucleotide handling enzyme is preferably derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are polymerases, exonucleases, helicases, translocases and topoisomerases, such as gyrases. Suitable enzymes include, but are not limited to, exonuclease I from E. coli (SEQ ID NO: 11), exonuclease III enzyme from E. coli (SEQ ID NO: 13), RecJ from T. thermophilus (SEQ ID NO: 15) and bacteriophage lambda exonuclease (SEQ ID NO: 17), TatD exonuclease and variants thereof. Three subunits comprising the sequence shown in SEQ ID NO: 15 or a variant thereof interact to form a trimer exonuclease. The polymerase may be PYROPHAGE® 3173 DNA Polymerase (which is commercially available from LUCIGEN® Corporation), SD Polymerase (commercially available from BIORON®) or variants thereof. The enzyme is preferably Phi29 DNA polymerase (SEQ ID NO: 9) or a variant thereof. The topoisomerase is preferably a member of any of the Moiety Classification (EC) groups 5.99.1.2 and 5.99.1.3.

The enzyme is most preferably derived from a helicase. The helicase may be or be derived from a He1308 helicase, a RecD helicase, such as TraI helicase or a TrwC helicase, a XPD helicase or a Dda helicase. The helicase may be or be derived from He1308 Mbu (SEQ ID NO: 18), He1308 Csy (SEQ ID NO: 19), He1308 Tga (SEQ ID NO: 20), He1308 Mhu (SEQ ID NO: 21), TraI Eco (SEQ ID NO: 22), XPD Mbu (SEQ ID NO: 23) or a variant thereof.

The helicase may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013098561); PCT/GB2013/051925 (published as WO 2014/013260); PCT/GB2013/051924 (published as WO 2014/013259); PCT/GB2013/051928 (published as WO 2014/013262) and PCT/GB2014/052736 (published as WO/2015/055981).

The helicase preferably comprises the sequence shown in SEQ ID NO: 25 (Trwc Cba) or as variant thereof, the sequence shown in SEQ ID NO: 18 (He1308 Mbu) or a variant thereof or the sequence shown in SEQ ID NO: 24 (Dda) or a variant thereof. Variants may differ from the native sequences in any of the ways discussed below for transmembrane pores. A preferred variant of SEQ ID NO: 24 comprises (a) E94C and A360C or (b) E94C, A360C, C109A and C136A and then optionally (ΔM1) G1 (i.e. deletion of M1 and then addition G1). It may also be termed M1G. Any of the variants discussed above may further comprise M1G.

The Dda helicase preferably comprises any of the modifications disclosed in International Application Nos. PCT/GB2014/052736 and PCT/GB2015/052916 (published as WO/2015/055981 and WO 2016/055777).

Any number of helicases may be used in accordance with the invention. For instance, 1, 2, 3, 4, 5, 6, 7, 8, 9, 10 or more helicases may be used. In some embodiments, different numbers of helicases may be used.

The method of the invention preferably comprises contacting the polynucleotide with two or more helicases. The two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

A variant of SEQ ID NO: 9, 11, 13, 15, 17, 18, 19, 20, 21, 22, 23, 24 or 25 is an enzyme that has an amino acid sequence which varies from that of SEQ ID NO: 9, 11, 13, 15, 17, 18, 19, 20, 21, 22, 23, 24 or 25 and which retains polynucleotide binding ability. This can be measured using any method known in the art. For instance, the variant can be contacted with a polynucleotide and its ability to bind to and move along the polynucleotide can be measured. The variant may include modifications that facilitate binding of the polynucleotide and/or facilitate its activity at high salt concentrations and/or room temperature. Variants may be modified such that they bind polynucleotides (i.e. retain polynucleotide binding ability) but do not function as a helicase (i.e. do not move along polynucleotides when provided with all the necessary components to facilitate movement, e.g. ATP and Mg²⁺). Such modifications are known in the art. For instance, modification of the Mg²⁺ binding domain in helicases typically results in variants which do not function as helicases. These types of variants may act as molecular brakes (see below).

Over the entire length of the amino acid sequence of SEQ ID NO: 9, 11, 13, 15, 17, 18, 19, 20, 21, 22, 23, 24 or 25, a variant will preferably be at least 50% homologous to that sequence based on amino acid simlarity or identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid simlarity or identity to the amino acid sequence of SEQ ID NO: 9, 11, 13, 15, 17, 18, 19, 20, 21, 22, 23, 24 or 25 over the entire sequence. There may be at least 80%, for example at least 85%, 90% or 95%, amino acid simlarity or identity over a stretch of 200 or more, for example 230, 250, 270, 280, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NO: 2 and 4 above. The enzyme may be covalently attached to the pore. Any method may be used to covalently attach the enzyme to the pore.

A preferred molecular brake is TrwC Cba-Q594A (SEQ ID NO: 25 with the mutation Q594A). This variant does not function as a helicase (i.e. binds polynucleotides but does not move along them when provided with all the necessary components to facilitate movement, e.g. ATP and Mg²⁺).

In strand sequencing, the polynucleotide is translocated through the pore either with or against an applied potential. Exonucleases that act progressively or processively on double stranded polynucleotides can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

Any helicase may be used in the method. Helicases may work in two modes with respect to the pore. First, the method is preferably carried out using a helicase such that it moves the polynucleotide through the pore with the field resulting from the applied voltage. In this mode the 5′ end of the polynucleotide is first captured in the pore, and the helicase moves the polynucleotide into the pore such that it is passed through the pore with the field until it finally translocates through to the trans side of the membrane. Alternatively, the method is preferably carried out such that a helicase moves the polynucleotide through the pore against the field resulting from the applied voltage. In this mode 3′ end of the polynucleotide is first captured in the pore, and the helicase moves the polynucleotide through the pore such that it is pulled out of the pore against the applied field until finally ejected back to the cis side of the membrane.

The method may also be carried out in the opposite direction. The 3′ end of the polynucleotide may be first captured in the pore and the helicase may move the polynucleotide into the pore such that it is passed through the pore with the field until it finally translocates through to the trans side of the membrane.

When the helicase is not provided with the necessary components to facilitate movement or is modified to hinder or prevent its movement, it can bind to the polynucleotide and act as a brake slowing the movement of the polynucleotide when it is pulled into the pore by the applied field. In the inactive mode, it does not matter whether the polynucleotide is captured either 3′ or 5′ down, it is the applied field which pulls the polynucleotide into the pore towards the trans side with the enzyme acting as a brake. When in the inactive mode, the movement control of the polynucleotide by the helicase can be described in a number of ways including ratcheting, sliding and braking. Helicase variants which lack helicase activity can also be used in this way.

The polynucleotide may be contacted with the polynucleotide binding protein and the pore in any order. It is preferred that, when the polynucleotide is contacted with the polynucleotide binding protein, such as a helicase, and the pore, the polynucleotide firstly forms a complex with the protein. When the voltage is applied across the pore, the polynucleotide/protein complex then forms a complex with the pore and controls the movement of the polynucleotide through the pore.

Any steps in the method using a polynucleotide binding protein are typically carried out in the presence of free nucleotides or free nucleotide analogues and an enzyme cofactor that facilitates the action of the polynucleotide binding protein. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

The molecular brakes may be any compound or molecule which binds to the polynucleotide and slows the movement of the polynucleotide through the pore. The molecular brake may be any of those discussed above. The molecular brake preferably comprises a compound which binds to the polynucleotide. The compound is preferably a macrocycle. A macrocycle is a cyclic macromolecule or a macromolecular cyclic portin of a molecule. The macrocycle may comprise a ring having an inner circumference made up of a chain of at least 12 connections or atoms, up to 100, 500 or more connections or atoms. The inner diameter of the macrocycle ring (Van der Waals radius) may be in the range of from about 0.5 nm to less than about 10 nm, preferably in the range of from 1 nm to 3 nm. Suitable macrocycles include, but are not limited to, cyclodextrins, calixarenes, cyclic peptides, crown ethers, cucurbiturils, pillararenes, derivatives thereof or a combination thereof. The cyclodextrin or derivative thereof may be any of those disclosed in Eliseev, A. V., and Schneider, H-J. (1994) J. Am. Chem. Soc. 116, 6081-6088. The cyclodextrin is more preferably heptakis-6-amino-β-cyclodextrin (am₇-βCD), 6-monodeoxy-6-monoamino-β-cyclodextrin (am₁-βCD) or heptakis-(6-deoxy-6-guanidino)-cyclodextrin (gu₇-βCD).

### Spacers in the Target Polynucleotide

If a helicase is used in the invention, it may be stalled at one or more spacers as discussed in International Application No. PCT/GB2014/050175 (published as WO 2014/135838). Any configuration of one or more helicases and one or more spacers disclosed in the International Application may be used in this invention.

### Double Stranded Polynucleotide

If the polynucleotide is double stranded, the method preferably further comprises providing the polynucleotide with a hairpin loop at one end of the polynucleotide. The method may comprise linking the two strands of the polynucleotide at one end with a hairpin loop. The molecular brake preferably separates the two strands of the target polynucleotide and controls the movement of the target polynucleotide through the pore one strand at a time. Linking and interrogating both strands on a double stranded construct in this way increases the efficiency and accuracy of characterisation. Hairpin loops are discussed above.

### Leader Sequence

The polynucleotide may be provided with a leader sequence which preferentially threads into the pore. The leader sequence facilitates the method of the invention. The leader sequence is designed to preferentially thread into the transmembrane pore and thereby facilitate the movement of polynucleotide through the pore. The leader sequence can also be used to link the polynucleotide to the one or more anchors as discussed above.

The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The leader sequence can comprise any of the polynucleotides discussed above. The single stranded leader sequence most preferably comprises a single strand of DNA, such as a poly dT section. The leader sequence preferably comprises the one or more spacers.

The leader sequence can be any length, but is typically 10 to 150 nucleotides in length, such as from 20 to 150 nucleotides in length. The length of the leader typically depends on the transmembrane pore used in the method.

### Y Adaptors

A double stranded polynucleotide may be provided with a Y adaptor at one end and a hairpin loop at the other end. The method of the invention may comprise attaching a Y adaptor to one end of a double stranded polynucleotide and attaching a hairpin loop at the other end. The Y adaptor and/or the hairpin adaptor are typically polynucleotide adaptors. They may be formed from any of the polynucleotides discussed above.

The Y adaptor typically comprises (a) a double stranded region and (b) a single stranded region or a region that is not complementary at the other end. The Y adaptor may be described as having an overhang if it comprises a single stranded region. The presence of a non-complementary region in the Y adaptor gives the adaptor its Y shape since the two strands typically do not hybridise to each other unlike the double stranded portion. The Y adaptor may comprise one or more anchors. Anchors are discussed in more detail above.

The Y adaptor preferably comprises a leader sequence which preferentially threads into the pore. Leader sequences are discussed above.

The Y adaptor preferably comprises a selectable binding moiety as discussed above. The Y adaptor and/or the selectable binding moiety may comprise a region that can be cut, nicked, cleaved or hydrolysed as discussed above.

The Y adaptor and/or the hairpin loop may be ligated to the polynucleotide using any method known in the art. One or both of the adaptors may be ligated using a ligase, such as T4 DNA ligase, E. coli DNA ligase, Taq DNA ligase, Tma DNA ligase and 9°N DNA ligase. Alternatively, the adaptors may be added to the polynucleotide using the methods of the invention discussed below.

In a preferred embodiment, the method comprises modifying the double stranded polynucleotide so that it comprises the Y adaptor at one end and the hairpin loop at the other end. Any manner of modification can be used. The method preferably comprises modifying the double stranded polynucleotide in accordance with the invention. This is discussed in more detail below. The methods of modification and characterisation may be combined in any way.

### Adding Hairpin Loops and Leader Sequences

The double stranded polynucleotide may be provided with Y and hairpin loops by contacting the polynucleotide with a MuA transposase and a population of double stranded MuA substrates, wherein a proportion of the substrates in the population are Y adaptors comprising the leader sequence and wherein a proportion of the substrates in the population are hairpin loops. The transposase fragments the double stranded polynucleotide and ligates MuA substrates to one or both ends of the fragments. This produces a plurality of modified double stranded polynucleotides comprising the leader sequence at one end and the hairpin loop at the other. The modified double stranded polynucleotides may then be investigated using the method of the invention.

These MuA based methods are disclosed in International Application No. PCT/GB2014/052505 published as (WO 2015/022544). They are also discussed in detail in International Application No PCT/GB2015/050991.

### Modified Polynucleotide Analytes

Before characterisation in accordance with the invention, the polynucleotide may be modified by contacting the polynucleotide with a polymerase and a population of free nucleotides under conditions in which the polymerase forms a modified polynucleotide using the polynucleotide as a template, wherein the polymerase replaces one or more of the nucleotide species in the polynucleotide with a different nucleotide species when forming the modified polynucleotide analyte. The modified polynucleotide may then be characterised in accordance with the invention. This type of modification is described in PCT Application No. PCT/GB2015/050483. Any of the polymerases discussed above may be used. The polymerase is preferably Klenow or 90 North.

### Kits

The present invention also provides a kit for characterising a double stranded target polynucleotide. The kit comprises a hairpin loop capable of linking the two strands of the target polynucleotide at one end. Such loops are discussed above. The kit also comprises one or more species which control or decrease the formation of secondary structure by the target polynucleotide. Any of the species discussed above may be present in the kit.

The kit may further comprise a microparticle for delivering the polynucleotide to a transmembrane pore in a membrane. The kit may further comprise one or more anchors which are capable of coupling the polynucleotide to a membrane. The microparticle and one or more anchors may be any of those discussed above with reference to the method of the invention. The microparticle is preferably part of the kit for extracting and/or purifying the polynucleotide.

The kit preferably further comprises a leader sequence which is capable of preferentially threading into a transmembrane pore. The kit may comprise a Y adaptor. The kit preferably further comprises a molecular brake, such as a polynucleotide binding protein. Preferred leader sequences, Y adaptors, molecular brakes and polynucleotide binding proteins are discussed above.

Any of the embodiments discussed above with reference to the method of the invention equally apply to the kits. The kit may further comprise the components of a membrane, such as the components of an amphiphilic layer or a triblock copolymer membrane. The kit may further comprise a transmembrane protein pore.

The kit of the invention may additionally comprise one or more other reagents or instruments which enable any of the embodiments mentioned above to be carried out. The kit may comprise a magnet or an electromagnet. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used.

### Apparatus

The invention also provides an apparatus for characterising a target polynucleotide. The apparatus comprises a plurality of membranes and a plurality of pores. The plurality of pores is present in the plurality of membranes. The number of pores and membranes is preferably equal. Preferably, a single pore is present in each membrane.

The apparatus also comprises conditions on the other side of the pores from which the pores are contacted with the polynucleotide which are capable of controlling the formation of secondary structure by the target polynucleotide. Any of the conditions discussed above may be present in the apparatus.

The apparatus preferably comprises a plurality of molecular brakes on the side of the pores which is contacted with the polynucleotide and which are capable of controlling the movement of the target polynucleotide through the pores. Suitable molecular brakes are discussed above.

The apparatus preferably further comprises instructions for carrying out the method of the invention. The apparatus may be any conventional apparatus for analyte analysis, such as an array or a chip. Any of the embodiments discussed above with reference to the methods of the invention are equally applicable to the apparatus of the invention. The apparatus may further comprise any of the features present in the kit of the invention.

The apparatus is preferably set up to carry out the method of the invention.

The apparatus preferably comprises:
a sensor device that is capable of supporting the plurality of pores and membranes and being operable to perform analyte characterisation using the pores and membranes; and
at least one port for delivery of the material for performing the characterisation.

Alternatively, the apparatus preferably comprises:
a sensor device that is capable of supporting the plurality of pores and membranes being operable to perform analyte characterisation using the pores and membranes; and at least one reservoir for holding material for performing the characterisation.

The apparatus more preferably comprises:
a sensor device that is capable of supporting the membrane and plurality of pores and membranes and being operable to perform analyte characterising using the pores and membranes;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device.

The apparatus may be any of those described in International Application No. No. PCT/GB08/004127 (published as WO 2009/077734), PCT/GB10/000789 (published as WO 2010/122293), International Application No. PCT/GB10/002206 (published as WO 2011/067559) or International Application No. PCT/US99/25679 (published as WO 00/28312).

A system is also provided which comprises: (a) a transmembrane pore in a membrane, wherein the membrane defines a first side and a second side, wherein the second side provides a condition that controls formation of a secondary structure from a portion of the target polynucleotide that moves through the transmembrane pore to the second side. The condition that controls formation of a secondary structure from a portion of the target polynucleotide may be any of the conditions described herein for that purpose.

The following Examples illustrate the invention.

### EXAMPLES

### Example 1

This example illustrates how the formation of secondary structure by the target polynucleotide was controlled by the addition of a number of different polynucleotide sequences to the opposite side of the pore (trans side) to which the target polynucleotide was contacted (cis side).

### Materials and Methods

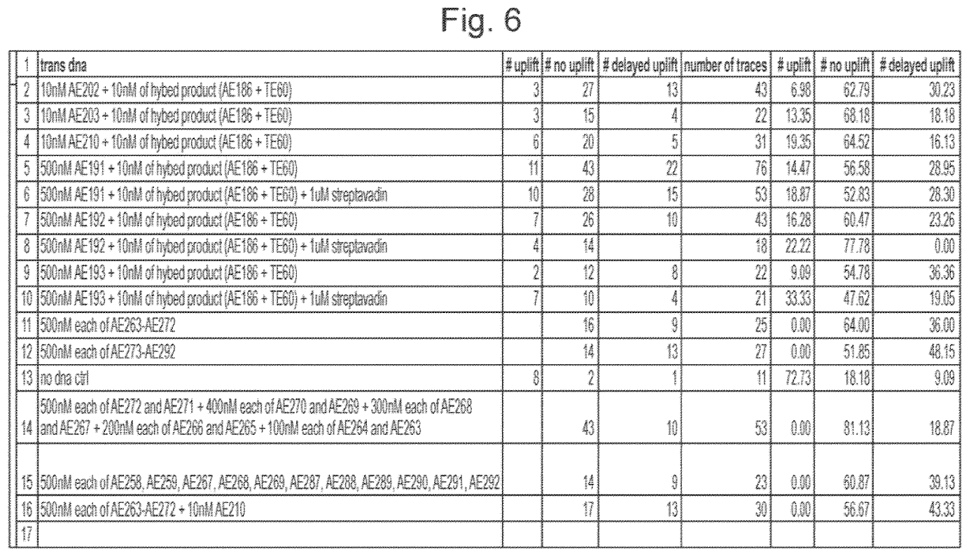

The experimental setup initially consisted of buffer 1 (600 mM KCl, 25 mM K Phosphate buffer, 75 mM Potassium Ferrocyanide (II), 25 mM Potassium Ferricyanide (III), pH 8.0) in the cis and trans sides. The trans side also contained additional additives depending on the experiment e.g. DNA (which may have had a biotin and/or cholesterol modification present) alone or DNA and streptavidin (see table in FIG. 6 for details and description of the DNA sequences for the sequences of the DNA polynucleotides added to the trans side of the nanopore).

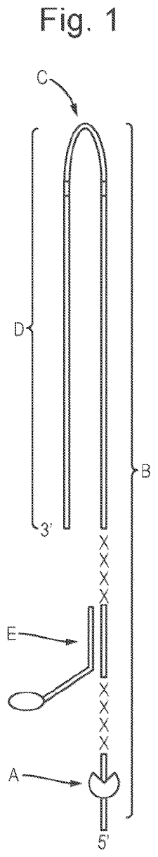

Electrical measurements were acquired from single MspA nanopores inserted in block co-polymer in buffer 1. After achieving a single pore inserted in the block co-polymer, then 1 mL of buffer 1 was flowed through the system to remove any excess MspA nanopores. 300 μL of buffer 1 with 0.1 nM construct 1 (see description of DNA sequences and FIG. 1 for the sequence of construct 1), 10 mM MgCl2, 1 mM ATP was then flowed into the single nanopore experimental system. The experiment was run at −120 mV and helicase-controlled DNA movement was monitored.

### Description of the DNA Sequences

DNA sequence 1 is shown in SEQ ID NO: 28.
DNA Sequence 2 is shown in SEQ ID NO: 29.
DNA sequence 3 is:

(SEQ ID NO: 32)
GCAATATCAGCACCAACAGAAACAACCT/iSp18//iSp18//
iSp18//iSp18//iSp18//iSp18/TT/3CholTEG/
DNA sequence 4 is:
(SEQ ID NO: 33)
CGTTCTGTTTATGTTTCTTGTTTGTTAGCCTTTTTTTTTTTTTT
TTTTT/iSpC3//iSpC3//iSpC3//iSpC3/
TTTTTGGCTAACAAACAAGAAACATAAACAGAACG
AE202 is:
(SEQ ID NO: 34)
/5Chol-TEG/IIIIIIIIII
AE186 is:
(SEQ ID NO: 35)
CAAATAAGAACATTATGATCAGTAGGGCTAACAAACAAGAAACATAAACA
GAACGTGCTTACGGTTCACTACTCACGACGATGTTTTTTTTGGTACCTTT
TTTTTCACCGGAAAG
TE60 is:
(SEQ ID NO: 36)
CTACTGATCATAATGTTCTTATTTGT/iSp18//iSp18//iSp18//
iSp18//iSp18//iSp18/TT/3CholTEG/
AE203 is:
(SEQ ID NO: 38)
/5Chol-TEG/IIIIIIIIIIIIIIIIIIII
AE210 is:
(SEQ ID NO: 39)
/5BiodT/IIIIIIIIIIIIIIIIIIIIIIIIIIIIII/
3CholTEG/
AE191 is:
(SEQ ID NO: 40)
/5BiodT/IIIIIIIIII
AE192 is:
(SEQ ID NO: 41)
/5BiodT/IIIIIIIIIIIIIIIIIIII
AE193 is:
(SEQ ID NO: 42)
/5BiodT/IIIIIIIIIIIIIIIIIIIIIIIIIIIIII

AE263 is shown in SEQ ID NO: 43.
AE264 is shown in SEQ ID NO: 44.
AE265 is shown in SEQ ID NO: 45.
AE266 is shown in SEQ ID NO: 46.
AE267 is shown in SEQ ID NO: 47.
AE268 is shown in SEQ ID NO: 48.
AE269 is shown in SEQ ID NO: 49.
AE270 is shown in SEQ ID NO: 50.
AE271 is shown in SEQ ID NO: 51.
AE272 is shown in SEQ ID NO: 52.
AE273 is shown in SEQ ID NO: 53.
AE274 is shown in SEQ ID NO: 54.
AE275 is shown in SEQ ID NO: 55.
AE276 is shown in SEQ ID NO: 56.
AE277 is shown in SEQ ID NO: 57.
AE278 is shown in SEQ ID NO: 58.
AE279 is shown in SEQ ID NO: 59.
AE280 is shown in SEQ ID NO: 60.
AE281 is shown in SEQ ID NO: 61.
AE282 is shown in SEQ ID NO: 62.
AE283 is shown in SEQ ID NO: 63.
AE284 is shown in SEQ ID NO: 64.
AE285 is shown in SEQ ID NO: 65.
AE286 is shown in SEQ ID NO: 66.
AE287 is shown in SEQ ID NO: 67.
AE288 is shown in SEQ ID NO: 68.
AE289 is shown in SEQ ID NO: 69.
AE290 is shown in SEQ ID NO: 70.
AE291 is shown in SEQ ID NO: 71.
AE292 is shown in SEQ ID NO: 72.
AE258 is shown in SEQ ID NO: 73.
AE259 is shown in SEQ ID NO: 74.
AE182 is shown in SEQ ID NO: 75.

### Analysis

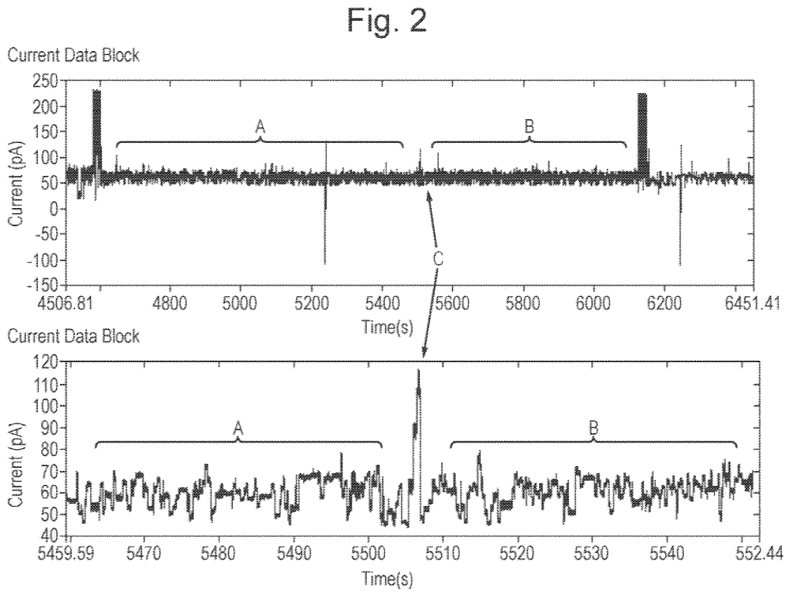

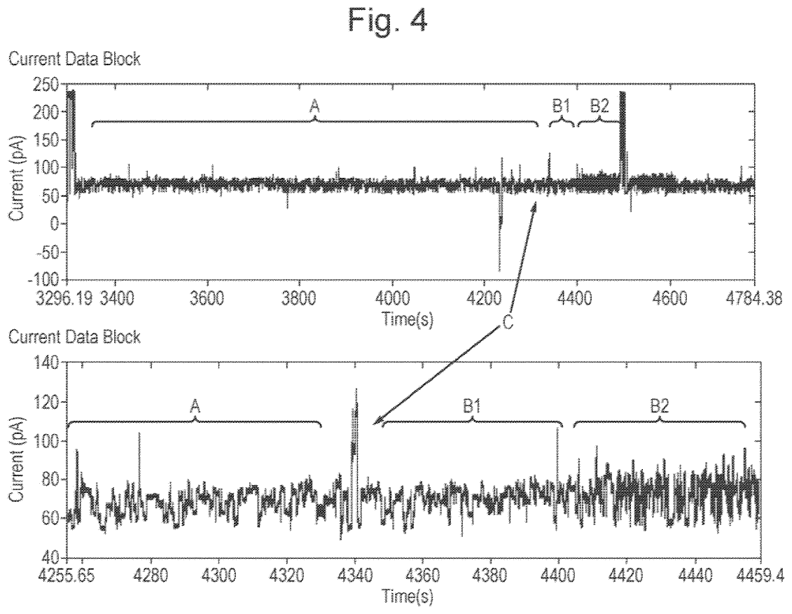

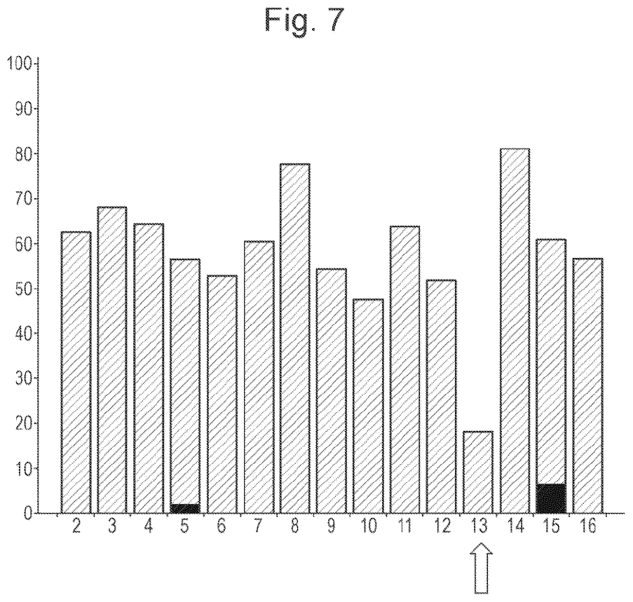

For the experiments in which there was DNA in the trans, the signals produced by construct 1 translocating through the pore in which the hairpin was clearly identified were observed and categorized as either having uplift, no uplift or delayed uplift (FIGS. 2-4) this was done with several different trans DNA sequences and concentrations. This data was compiled into a table showing what was in the trans and number of helicase controlled polynucleotide movements in each category (see FIG. 6). This data was also plotted as a bar chart (see FIG. 7).

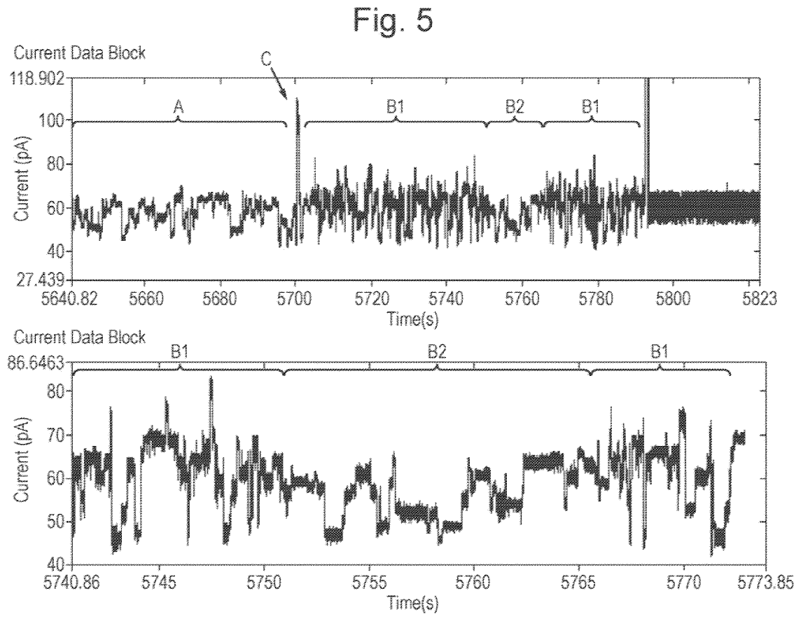

In addition, experiments were also carried out with DNA sequence AE182 in the trans at a concentration of 500 nM in buffer 1. For these experiments the signals were observed to see if they had the expected non-uplifted section in a background of uplifted complement (FIG. 5).

### Results

The data shown in FIGS. 6 and 7 illustrates that the addition of a variety of different polynucleotide sequences (see Col 1 of FIG. 6) which hybridise to construct 1 successfully reduced the number of helicase controlled DNA movements which exhibited uplift in the complement region of construct 1. The control condition (no addition of any polynucleotide sequences shown in row 13 of FIGS. 6 and 7) exhibited approximately 80% uplift in the helicase controlled DNA movements. Whereas conditions 2-12 and 14-16 all exhibited a decrease in the % of uplift observed in the helicase controlled DNA movements. Therefore, the addition of the polynucleotide sequences to the trans side (the opposite side of the pore to which the target polynucleotide was contacted) reduced the formation of secondary structure by the target polynucleotide sequence on the trans side of the nanopore.

### Example 2

This example illustrates how the formation of secondary structure by the target polynucleotide was controlled by the addition of an exonuclease (SAN) to the opposite side of the pore (trans side) to which the target polynucleotide was contacted (cis side).

### Materials and Methods

The experimental setup initially consisted of buffer 1 (600 mM KCl, 25 mM K Phosphate buffer, 75 mM Potassium Ferrocyanide (II), 25 mM Potassium Ferricyanide (III), pH 8.0) in the cis and trans. The trans side also contains 10 mM MgCl2 and 0.005 U/uL and 0.1 U/uL Salt Activated Nuclease (SAN) from Arcticzymes.

Electrical measurements were acquired from single MspA nanopores inserted in block co-polymer in buffer 1. After achieving a single pore inserted in the block co-polymer, then 1 mL of buffer 1 was flowed through the system to remove any excess MspA nanopores. 300 μL of buffer 1 with 0.1 nM construct 1 (see description of DNA sequences and FIG. 1 for the sequence of construct 1), 10 mM MgCl2, 1 mM ATP was then flowed into the single nanopore experimental system. The experiment was run at −120 mV and helicase-controlled DNA movement monitored.

### Analysis

Analysis was carried out for this experiment in the same way as for Example 1 described previously.

### Results

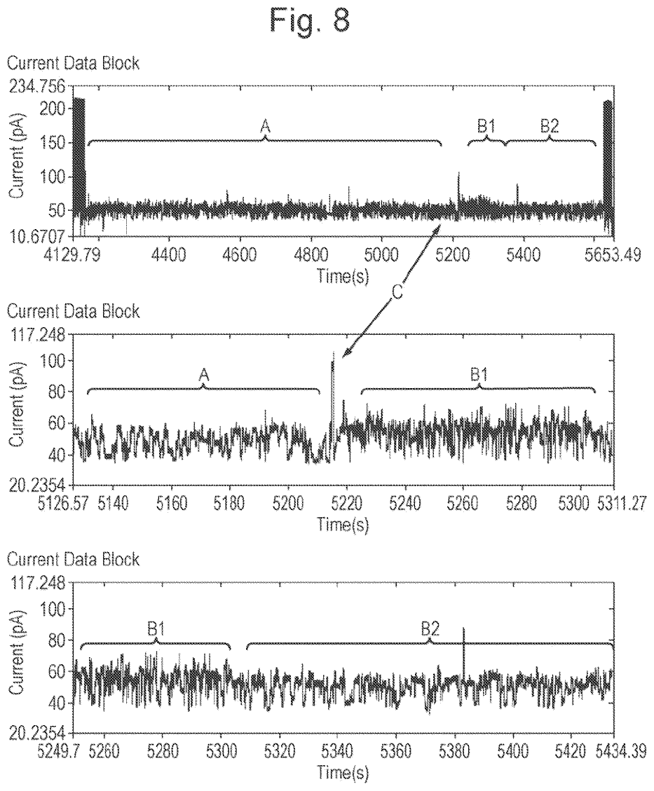

For experiments for which there was SAN in trans, the helicase controlled DNA movements were observed to see if there was any uplift. 50-60% of the helicase controlled DNA movements were observed to have no uplift which is higher than the 20% no uplift observed for the control detailed in example 1. Helicase controlled DNA movements of the type shown in FIG. 2 (non-uplifted), FIG. 3 (uplifted) and a new type of behaviour such as that shown in FIG. 8 (initial uplift in the complement strand region and then becoming non-uplifted-which indicates cleavage by SAN) were observed in this experiment. Therefore, the addition of SAN to the trans side (the opposite side of the pore to which the target polynucleotide was contacted) reduced the formation of secondary structure by the target polynucleotide sequence on the trans side of the nanopore.

### Example 3

This example illustrates how the hairpin loop is designed to control the ability of the two strands of the target polynucleotide to rehybridise on the other side of the pore. G-Quadruplex (G4) are ribonucleic acid secondary structures. Incorporating them in to hairpins offers one way of attenuating uplift behaviour by providing the DNA with an alternative to re-hybridisation.

A useful property of G4 is their tendency to form stable structures in certain ionic environments, meaning the hairpin structures may be formed in line with existing methods. The G4 structure is “activated” once the hairpin has been broken (ie stripped by the nanopore revealing the G4 sequence) and in the presence of a high concentration of suitable ions.

### DNA Sequences

| | |
|---|---|
| TH14 | /5Phos/CGT GGTTGGTGTGGTTGG CG |
| | GACACTGATTGACACGGTTTAGTAGAGC/iSp18//iSp18// |
| | iSp18//iSp18/TTTTTTTTTTTTTTTTTTTTTTTCGCCAAC |
| | CACACCAACCACGTCCT (SEQ ID NO: 76) |
| TH15 | /5Phos/CGT CCAACCACACCAACC CGGACACTGATTGAC |
| | ACGGTTTAGTAGAGC/iSp18//iSp18//iSp18//iSp18/ |
| | TTTTTTTTTTTTTTTTTTTTTTTCGGGTTGGTGTGGTTGG |
| | ACGTCCT (SEQ ID NO: 77) |
| TH16 | /5Phos/CGT AGTCCGTGGTAGGGCAGGTTGGGGTGACCGGA |
| | CACTGATTGACACGGTTTAGTAGAGC/iSp18//iSp18// |
| | iSp18//iSp18/TTTTTTTTTTTTTTTTTTTTTTTCGAGT |
| | CACCCCAACCTGCCCTACCACGGACTACGTCCT (SEQ ID |
| | NO: 78) |
| TH17 | /5Phos/CGTAGTCACCCCAACCTGCCCTACCACGGACTCGGA |
| | CACTGATTGACACGGTTTAGTAGAGC/iSp18//iSp18// |
| | iSp18//iSp18/TTTTTTTTTTTTTTTTTTTTTTTCGA |
| | GTCCGTGGTAGGGCAGGTTGGGGTGACT ACGTCCT (SEQ |
| | ID NO: 79) |

TH14 and 15 contain TBA sequence highlighted.
TH16 and 17 contain a TBA derivative called HD22 highlighted. This contains a psudeo G4 with additional duplex structure.

Control hairpin sequence =
(SEQ ID NO: 80)
/5Phos/CGTCCTGTCGCTGTGTCTCGGACACTGATTGACACGGTTTAGT
AGAGC/iSpC3//iSpC3//iSpC3//iSpC3/TTTTTTTTTTTTTTTTT
TTTTTTTTTTTCGAGACACAGCGACAGGACGTCCT
Y adapter sequence 1
(SEQ ID NO: 81)
5SpC3//iSpC3//iSpC3//iSpC3//iSpC3//iSpC3//iSpC3//
iSpC3//iSpC3//iSpC3//iSpC3//iSpC3//iSpC3//iSpC3//
iSpC3//iSpC3//iSpC3//iSpC3//iSpC3//iSpC3//iSpC3//
iSpC3//iSpC3//iSpC3//iSpC3//iSpC3//iSpC3//iSpC3//
iSpC3//iSpC3/GGCGTCTGCTTGGGTGTTTAACCTTTTTTTTTTTT/
iSp18//iSp18//iSp18//iSp18/GGTTGTTTCTGTTGGTGCTG
ATATTGCT
Y adapter sequence 2
(SEQ ID NO: 82)
5Phos/GCAATATCAGCACCAACAGAAA/iBNA-MeC//iBNA-
A//iBNA-A//iBNA-MeC//iBNA-MeC/TTTGAGGCGAGCGGTCAA
Y adapter sequence 3
(SEQ ID NO: 83)
/5BNA-G//iBNA-G//iBNA-T//iBNA-T//iBNA-A/AACACCCAAG
CAGACGCCTT

### Method

Oligos were stocked at 100 uM in TE pH 8.0. The oligos were then diluted to 500 nM in 10 mM Tris, 2 mM EDTA, 50 mM NaCl and heated at 95° C. for 15 min. The diluted oligos were then transferred to an ice bath for 15 min.

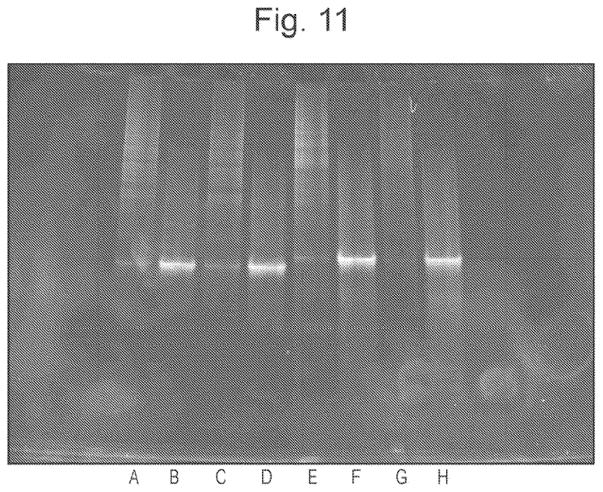

Ing of stock was run against Ing of snapcooled DNA on a 4-20% TBE gel (2ul DNA+4 ul native sample buffer, 4-20% TBE gel (see FIG. 11), TBE running buffer, 100 mV 1 hr, 160 mV 30 min. TBE/Sybr Gold 5 min, H₂O 5 min). A single band in the gel shown in FIG. 11 suggests the oligonucleotide is a single species.

The Y-adapter sequences were annealed. The Y-adapter and the appropriate hairpin adapter (TH14, 15, 16 or 17) were then ligated to dsDNA. A helicase to control the DNA movement was also loaded onto the DNA. This was known as DNA construct produced by ligation.

Electrical measurements were acquired from single MspA nanopores inserted in block co-polymer in buffer (25 mM K Phosphate buffer, 150 mM Potassium Ferrocyanide (II), 150 mM Potassium Ferricyanide (III), pH 8.0). After achieving a single pore inserted in the block co-polymer, then buffer (2 mL, 25 mM K Phosphate buffer, 150 mM Potassium Ferrocyanide (II), 150 mM Potassium Ferricyanide (III), pH 8.0) was flowed through the system to remove any excess MspA nanopores. 150 μL of 500 mM KCl, 25 mM K Phosphate, pH8.0 was then flowed through the system. After 10 minutes a further 150 μL of 500 mM KCl, 25 mM K Phosphate, pH8.0 was flowed through the system and then the enzyme (see list below, 10 nM final concentration), DNA construct produced by ligation (see previous method) (0.1 nM final concentration), fuel (MgCl2 2 mM final concentration, ATP 2 mM final concentration) pre-mix (150 uL total) was then flowed into the single nanopore experimental system. The experiment was run at −140 mV and helicase-controlled DNA movement monitored.

### Results

The DNA will face range of other ionic environments before it meets the trans-side of a nanopore (ligation buffer, wash buffers, running buffers etc). The structure remained as a hairpin at least until ligation had occurred. PAGE analysis suggests the hairpin confirmation is stable and preferred. The hairpin structure was preferred to G4 until denatured by the nanopore.

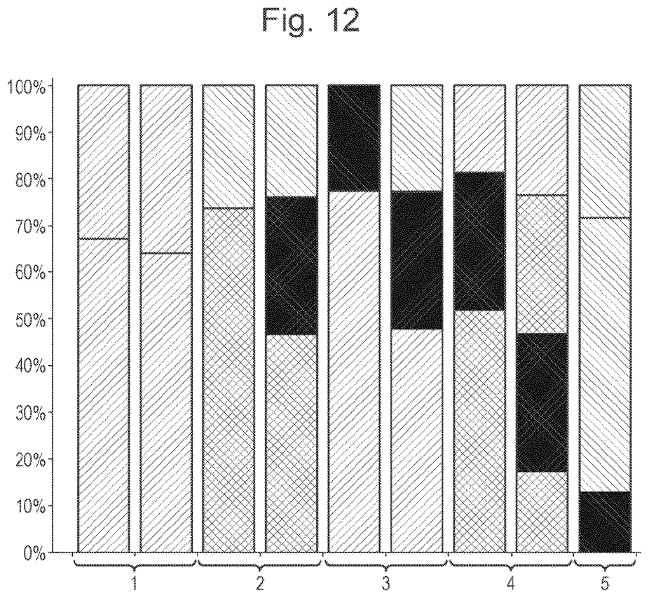

As shown in FIG. 12, all G4 designs (TH14-TH17) showed an improved proportion of non-uplifted to uplifted. Therefore, the use of hairpins containing G4 controlled the ability of the two strands of the target polynucleotide to rehybridise on the other side of the pore.

1-28. (canceled)

29. A method of characterising a target polynucleotide, comprising:
(a) providing a transmembrane pore in a membrane having a cis side and a trans side and carrying out steps (b)-(d) under one applied potential;
(b) contacting, on the cis side of the membrane, the target polynucleotide with the transmembrane pore and a molecular brake which controls the movement of the target polynucleotide through the transmembrane pore;
(c) taking one or more measurements as the molecular brake controls the movement of the target polynucleotide through the transmembrane pore, wherein the one or more measurements are indicative of one or more characteristics of the target polynucleotide as the target polynucleotide moves through the transmembrane pore; and
(d) chemically or physically cleaving a portion of the target polynucleotide that has translocated to the trans side of the membrane as the molecular brake controls movement of the target polynucleotide through the transmembrane pore, thereby decreasing formation of secondary structure by the target polynucleotide.

30. The method according to claim 29, wherein step (d) decreases formation of random secondary structure by the target polynucleotide on the trans side of the membrane, wherein the random secondary structure comprises (i) one or more helices, (ii) one or more loops, (iii) one or more pseudoknots, (iv) one or more quadruplexes, or (v) a combination thereof.

31. A method characterising a target polynucleotide, wherein the target polynucleotide is double stranded and the method comprises:
(a) providing a construct comprising the target polynucleotide in which the two strands of the target polynucleotide are linked at one end of the target polynucleotide by a hairpin loop;
(b) providing a transmembrane pore in a membrane having a cis side and a trans side and carrying out steps (c)-(e) under one applied potential;
(c) contacting, on the cis side of the membrane, the construct with the transmembrane pore and a molecular brake which separates the two strands of the construct and controls the movement of the construct through the transmembrane pore;
(d) taking one or more measurements as the molecular brake controls the movement of the construct through the transmembrane pore, wherein the one or more measurements are indicative of one or more characteristics of the target polynucleotide as the construct moves through the transmembrane pore; and
(e) chemically or physically cleaving a portion of the target polynucleotide that has translocated to the trans side of the membrane as the molecular brake controls movement of the construct through the transmembrane pore, thereby decreasing formation of secondary structure by the target polynucleotide.

32. The method according to claim 31, wherein the step (e) decreases rehybridization of the two strands of the target polynucleotide on the trans side of the membrane or prevent the two strands of the target polynucleotide from rehybridising on the trans side of the membrane.

33. The method according to claim 29, wherein the molecular brake is a polynucleotide binding protein.

34. The method according to claim 29, wherein the transmembrane pore is (i) a protein pore or (ii) a solid state pore.

35. The method according to claim 29, wherein the entire target polynucleotide moves through the transmembrane pore and is characterised.

36. The method according to claim 29, wherein characterising the target polynucleotide comprises estimating the sequence of the target polynucleotide or sequencing the target polynucleotide.

37. The method according to claim 29, wherein the molecular brake is a polymerase, helicase or exonuclease.

38. The method according to claim 29, wherein the transmembrane pore is a protein pore or based on Msp, α-hemolysin (α-HL), lysenin, CsgG, ClyA, Sp1 or FraC.

39. The method according to claim 29, wherein the target polynucleotide is single-stranded.

40. The method according to claim 31, wherein the molecular brake is a polynucleotide binding protein.

41. The method according to claim 31, wherein the transmembrane pore is (i) a protein pore or (ii) a solid state pore.

42. The method according to claim 31, wherein the entire target polynucleotide moves through the transmembrane pore and is characterised.

43. The method according to claim 31, wherein characterising the target polynucleotide comprises estimating the sequence of the target polynucleotide or sequencing the target polynucleotide.

44. The method according to claim 31, wherein the molecular brake is a polymerase, helicase or exonuclease.

45. The method according to claim 31, wherein the transmembrane pore is a protein pore or based on Msp, α-hemolysin (α-HL), lysenin, CsgG, ClyA, Sp1 or FraC.

46. The method according to claim 29, wherein step (d) comprises using a nuclease to cleave a portion of the target polynucleotide that has translocated to the trans side of the membrane as the molecular brake controls movement of the target polynucleotide through the transmembrane pore.

47. The method according to claim 46, wherein the nuclease is an endonuclease, an exonuclease or a uracil-specific excision reagent (USER).

48. The method according to claim 31, wherein step (e) comprises using a nuclease to cleave a portion of the target polynucleotide that has translocated to the trans side of the membrane as the molecular brake controls movement of the construct through the transmembrane pore.

49. The method according to claim 29, wherein the molecular brake is a helicase.

50. The method according to claim 31, wherein the molecular brake is a helicase.
